SEQUENCE LISTING (34 sequences)

SEQ ID NO: 1 - DNA, 360 bp, Capsicum annuum
  gene (1)...(360): Bs3 promoter
agcaaactctaatatatcatagtcaagctaacgaaacttatgcaagggaaatatgaaattagtatgcaagtaaactcaaagaactaatcattgaactgaaagatcaatatatcaaaaaaaaaaaaaaaacaataaaaccgtttaaccgatagattaaccatttctggttcagtttatgggttaaaccacaatttgcacaccctggttaaacaatgaacacgtttgcctgaccaattttattatataaacctaaccatcctcacaacttcaagttatcatcccctttctcttttctcctcttgttcttgtcacccgctaaatctatcaaaacacaagtagtcctagttgcacatatatttc

SEQ ID NO: 2 - DNA, 963 bp, Artificial Sequence
  Bs3 Super Promoter
ttagttttactttgaaatgcgaatgatacatgacacattagattgtacttgctttttaccacagatacaacgatacatttgtatatcttttcccttatagcaaactctaatatatcatagtcaagctaacgaaacttatgcaagggaaatatgaaattagtatgcaagtaaactcaaagaactaatcattgaactgaaagatcaatatatcaaaaaaaaaaaaaaacaataaaaccgtttaaccgatagattaaccatttctggttcagtttatgggttaaaccacaatttgcacaccctgaccggtttagttttactttgaaatgctataaacctcttttaccttgaatgatacatgacatatacacctcttttactcattagattgtactttacacacctcctaccacctctacttgctttttaccacagatctctatctcaaccccttttacaacgatacatttgtatacacctctttacattttatatcttttccctttatatacctacaccctatagcaaactctaattatttaccactcttaccttatatcatagtcaagctatatacctacactaccttaacgaaacttatgctacacacctcttttaataagggaaatatgaaatacacatctttaaaactttagtatgcaagtaatatatacctacactacactacctactcaaagaactaattacacattataccactcattgaactgaaagatataaatctcttttacctttcaatatatcaaaaatctctatataactccctttaaaaaaaaacaataactcgaggttaaacaatgaacacgtttgcctgaccaattttattatataaacctaaccatcctcacaacttcaagttatcatcccctttctcttttctcctcttgttcttgtcacccgctaaatctatcaaaacacaagtagtcctagttgcacatatatttc

SEQ ID NO: 3 - DNA, 422 bp, Artificial Sequence
  Bs3 short promoter
catagtcaagctaacgaaacttatgcaagggaaatatgaaattagtatgtataaacctcttttacctttatatacctacactacactaccttatacacctcttttacttataacaccctcaacataatcaagtaaactcaaagaactaatcattgaactgaaagatcaatatatcaaaaaaaaaaaaaaaacaataaaaccgtttaaccgatagattaaccatttctggttcagtttatgggttaaaccacaatttgcacaccctggttaaacaatgaacacgtttgcctgaccaattttattatataaacctaaccatcctcacaacttcaagttatcatcccctttctcttttctcctcttgttcttgtcacccgctaaatctatcaaaacacaagtagtcctagttgcacatatatttc

SEQ ID NO: 4 - protein, 532 aa, Xanthomonas axonopodis pv. citri
MAPSMHSAASPVSVLHLRDTSMRTKAQLPLTAIQRFLAHDAASTQAPSASASTSLHKNETAGLLAALPARNARQGAQRKSGEKEGARQNNGGRGGQWASRAAKYALGIAGAGYVADNFVLSTTSLVDGKGGFTSNDRLDKACAKAETYYARYHSATEDERASHSRPFVPIRTCGSNQFATMTDYRAATKVHVGHLFDSQAARESLVTNLACLKGERIKQECIIRYAPAQVPADPDLSKSELYDRKNKYSLVGMPNAQTGASGYTSRSITQPFINRGMEHFRQASQSDKALSLRQCMQSLERALQDTDKLGKQAQHAAGQAILNFRQVYAADEHWGHPEKVIMKTLIANGLLSQEQTDRIDATLMFEDPSISVLKRNTSIAGPLLQKLETKIQSKRLQDQPETLADFMEMAKQKNMEGLPIAHFKLNAEGTGFEDCSGLGDSFTSANAVACINHARLMSGEPRLSKEDVGVVVACLNAVYDDASSIRHSLHEIARGCFVGAGYTTEDADAFYEQICKDAARAFYAGKSMTSSD

SEQ ID NO: 5 - DNA, 19 bp, Xanthomonas citri subsp. citri
  misc_feature (1)...(19): UPT-Apl1
tataaacctcttttacctt

SEQ ID NO: 6 - DNA, 17 bp, Xanthomonas citri subsp. citri
  misc_feature (1)...(17): UPT-Apl2
tatacacctcttttact

SEQ ID NO: 7 - DNA, 25 bp, Xanthomonas citri subsp. citri
  misc_feature (1)...(25): UPT-Apl3
tacacacctcctaccacctctactt

SEQ ID NO: 8 - DNA, 19 bp, Xanthomonas fuscans subsp. aurantifoli
  misc_feature (1)...(19): UPT-PthB
tctctatctcaaccccttt

SEQ ID NO: 9 - DNA, 19 bp, Xanthomonas citri subsp. citri
  misc_feature (1)...(19): UPT-PthA*
tatacacctctttacattt

SEQ ID NO: 10 - DNA, 16 bp, Xanthomonas citri subsp. citri
  misc_feature (1)...(16): UPT-PthA*2
tatatacctacaccct

SEQ ID NO: 11 - DNA, 19 bp, Xanthomonas citri subsp. citri
  misc_feature (1)...(19): UPT-PthAw
tatttaccactcttacctt

SEQ ID NO: 12 - DNA, 18 bp, Xanthomonas citri subsp. citri
  misc_feature (1)...(18): UPT-PthA1
tatatacctacactacct

SEQ ID NO: 13 - DNA, 17 bp, Xanthomonas citri subsp. citri
  misc_feature (1)...(17): UPT-PthA2
tacacacctcttttaat

SEQ ID NO: 14 - DNA, 17 bp, Xanthomonas citri subsp. citri
  misc_feature (1)...(17): UPT-PthA3
tacacatctttaaaact

SEQ ID NO: 15 - DNA, 23 bp, Xanthomonas citri subsp. citri
  misc_feature (1)...(23): UPT-pB3.7
tatatacctacactacactacct

SEQ ID NO: 16 - DNA, 16 bp, Xanthomonas citri subsp. citri
  misc_feature (1)...(16): UPT-HssB3.0
tacacattataccact

SEQ ID NO: 17 - DNA, 19 bp, Xanthomonas citri subsp. citri
  misc_feature (1)...(19): UPT-PthA
tataaatctcttttacctt

SEQ ID NO: 18 - DNA, 19 bp, X. fuscans subsp. aurantifoli
  misc_feature (1)...(19): UPT-PthC
tctctatataactcccttt

SEQ ID NO: 19 - DNA, 20 bp, Xanthomonas citri subsp. citr
  misc_feature (1)...(20): UPT-AvrTAw
tataacaccctcaacataat

SEQ ID NO: 20 - protein, 36 aa, Artificial Sequence
  Repeat Variable Diresidues - PthA
NINGNINININGHDNGHDNGNGNGNGNSHDHDNGNG

SEQ ID NO: 21 - protein, 36 aa, Artificial Sequence
  Repeat Variable Diresidues - Apl1
NINGNININIHDHDNGHDNGNGNGNGNSHDHDNGNG

SEQ ID NO: 22 - protein, 36 aa, Artificial Sequence
  Repeat Variable Diresidues - PthAw
NINGNGNGNSHDHDNSHDNGNCNGNGNSHDHDNGNG

SEQ ID NO: 23 - protein, 36 aa, Artificial Sequence
  Repeat Variable Diresidues - PthA*
NINGNIHDNIHDHDNGHDNGNGNGNSHDNSNGNGNG

SEQ ID NO: 24 - protein, 44 aa, Artificial Sequence
  Repeat Variable Diresidues - pB3.7
NINGNINGNIHDHDNGNIHDNIHDNGNIHDNIHDNGNIHDHDNG

SEQ ID NO: 25 - protein, 34 aa, Artificial Sequence
  Repeat Variable Diresidues - PthA1
NINGNINGNIHDHDNGNIHDNIHDNGNIHDHDNG

SEQ ID NO: 26 - protein, 30 aa, Artificial Sequence
  Repeat Variable Diresidues - PthA*2
NINGNINGNIHDHDNGNIHDNIHDHDHDNG

SEQ ID NO: 27 - protein, 48 aa, Artificial Sequence
  Repeat Variable Diresidues - Apl3
NIHDNIHDNIHDHDNGHDHDNGNIHDHDNIHDHDNGHDHGHIHDNGNG

SEQ ID NO: 28 - protein, 32 aa, Artificial Sequence
  Repeat Variable Diresidues - Apl2
NINGNIHDNIHDHDNGHDNGNGNGNGNIHDNG

SEQ ID NO: 29 - protein, 32 aa, Artificial Sequence
  Repeat Variable Diresidues - PthA2
NIHDNIHDNIHDHDNGHDNGNGNGNGNINING

SEQ ID NO: 30 - protein, 32 aa, Artificial Sequence
  Repeat Variable Diresidues - PthA3
NIHDNIHDNINGHDNGNGNGNINININIHDNG

SEQ ID NO: 31 - protein, 30 aa, Artificial Sequence
  Repeat Variable Diresidues - HssB3.0
NIHDNIHDNINGNGNINGNIHDHDNIHDNG

SEQ ID NO: 32 - protein, 36 aa, Artificial Sequence
  Repeat Variable Diresidues - PthB
HDNGHDNGNINGHDNGHDNINIHDHDHDHDNGNGNG

SEQ ID NO: 33 - protein, 36 aa, Artificial Sequence
  Repeat Variable Diresidues - PthC
HDNGHDHDNINGNINGNINIHDNGHDHDHDNGNGNG

SEQ ID NO: 34 - protein, 38 aa, Artificial Sequence
  Repeat Variable Diresidues - avrTaw
NINGNINIHDNIHDHDHDNGHDNSNIHDNINGNINSNG

(19) United States
(12) Patent Application Publication
(10) Pub. No.: US 2014/0137292 A1
(43) Pub. Date: May 15, 2014

(54) CITRUS TREES WITH RESISTANCE TO CITRUS CANKER

(71) Applicants: Jeffrey B. Jones, Gainesville, FL (US); Thomas Lahaye, Stockdorf (DE); Brian J. Staskawicz, Berkeley, CA (US)

(72) Inventors: Jeffrey B. Jones, Gainesville, FL (US); Thomas Lahaye, Stockdorf (DE); Brian J. Staskawicz, Berkeley, CA (US)

(73) Assignees: UNIVERSITY OF FLORIDA RESEARCH FOUNDATION INC., Gainesville, FL (US); TWO BLADES FOUNDATION, Evanston, IL (US)

(21) Appl. No.: 13/976,703

(22) PCT Filed: Jan. 12, 2012
(86) PCT No.: PCT/US2012/021043

§ 371 Date: Oct. 11, 2013

Related U.S. Application Data
(60) Provisional application No. 61/433,192, filed on Jan. 14, 2011.
(60) Provisional application No. 61/433,929, filed on Jan. 18, 2011.

(51) Int. Cl. C12N 15/82

(52) U.S. Cl. CPC C12N 15/8281; C12N 15/8216
USPC 800/279; 800/301; 536/23.6; 435/320.1; 435/418

(57) ABSTRACT

Methods and compositions for making citrus plants with enhanced resistance to Asiatic citrus canker (ACC) and other forms of citrus canker caused by Xanthomonas are provided. The methods involve transforming citrus plant cells with polynucleotide constructs comprising a promoter operably linked to nucleotide sequence that encodes a protein that is capable of triggering cell death in a citrus plant. The promoters of the invention are inducible by one or more Xanthomonas strains that cause citrus canker. Isolated nucleic acid molecules and expression cassettes comprising such polynucleotide constructs and promoters are further provided. Citrus plants with enhanced resistance to citrus canker are also provided.

### FIELD OF THE INVENTION

This invention relates to the field of plant molecular biology, particularly the genetic improvement of plants through the use of methods involving recombinant DNA.

### BACKGROUND OF THE INVENTION

Deploying plant resistance has been the goal of many breeding programs to reduce losses resulting from plant diseases. With few exceptions, resistance is not commonly observed in citrus against Asiatic citrus canker (ACC). One example of a high level of tolerance to ACC was reported in Kumquat (Fortunella margarita); however, introgression of this resistance into widely grown citrus types such as sweet orange and grapefruit would be extremely difficult (Khalaf et al. (2008) Physiol. Mol. Plant Pathol. 71:240-250).

ACC adversely affects citrus production worldwide (Gottwald et al. (2002) Phytopathol. 92:361-77). The causal agents of ACC are the bacterial strains Xanthomonas citri subsp. citri (X. citri), also known as X. campestris pv citri, X, axonopodis pv citri, or X. smithii subsp citri, and X. fuscans subsp. Aurantifolii. These strains are part of a large group of Gram negative phytopatogenic bacteria that rely on a transmembrane needle-like structure known as the type III secretion system (T3SS) to inject an assortment of protein effectors into host mesophyll cells (Hogenhout et al. (2009) Mol. Plant-Microbe Interact. 22:115-122). In susceptible plants, these T3-effector proteins target host functions in order to shut down defense barriers and to promote a favorable environment for bacterial colonization (Zhou et al. (2008) Curr. Opin Microbiol. 11:179-185). Some plants have evolved resistance, and in such plants T3-effector proteins or their activities are specifically recognized by plant resistance (R) genes and R proteins, activating a program of defense responses that can culminate in a localized cell death reaction known as the hypersensitive response (HR; Büttner and Bonas (2010) FEMS MicrobioL Lett. 34:107-133).

One particular T3-effector class which is prominent in Xanthomonas species is the transcription activator-like (TAL) effectors, exemplified by AvrBs3 from X. euvesicatoria the causal agent of bacterial leaf spot in peppers. Following injection into the plant cell, TAL effectors translocate to the host cell nucleus and activate transcription through direct binding to DNA sequences in host promoters (Gürlebeck et al. (2005) Plant J. 42:175-187; Kay et al. (2007) Science 318:648-651; Wichmann and Bergelson (2004) Genetics 166: 693-706). As one example, the pepper (Capsicum annuum) cultivar Early California Wonder (ECW) is susceptible to X. euvesicatoria, which introduces AvrBs3 into host cells and activates UPA (UPregulated by AvrBs3) genes, such as UPA20 to promote hypertrophy (Kay et al. (2007) Science 318:648-651; Marois et al. (2002) Mol. Plant-Microbe Interact. 15:637-646). Other pepper cultivars, such as ECW-30R have evolved the resistance gene Bs3. The promoter of Bs3 also has an UPA recognition sequence (UPA box) and when activated by AvrBs3 triggers an HR (Marois et al. (2002) Mol. Plant-Microbe Interact. 15:637-646; Römer et al. (2007) Science 318: 645-648). The interaction of TAL effectors with DNA is mediated by specific amino acids in repeat domains in the central region of the protein. These amino acids, known as hypervariable residues or repeat variable diresidues (RVDs), directly contact bases in the target DNA sequences in a linear fashion according to a simple interaction code (Boch et al. (2009) Science 326:1509-1512; Moscou and Bogdanove (2009) Science 326:1501-1501). The target sequence is known as the UPregulated by AvrBs3, or UPA box, or more generally, as the UP-regulated by TAL effector, or UPT box, followed by a subscript designation of the particular TAL effector (Römer et al. (2009) PNAS 106:20526-20531).

### BRIEF SUMMARY OF THE INVENTION

Methods are provided for making a citrus plant with enhanced resistance to Asiatic citrus canker (ACC) and other citrus canker causing species of Xanthomonas. The methods involve transforming at least one citrus plant cell with a polynucleotide construct comprising a promoter operably linked to a coding sequence of an execution gene, wherein said promoter comprises at least one UPT box, and wherein said execution gene encodes an execution protein that is capable of triggering cell death in a citrus plant cell. The methods can further involve regenerating a transformed citrus plant from said citrus plant cell, wherein said transformed citrus plant comprises enhanced resistance to at least one Xanthomonas strain that causes citrus canker, particularly ACC. Preferably, the transformed citrus plants of the present invention have enhanced resistance to two or more Xanthomonas strains that cause citrus canker, particularly ACC.

In one embodiment of the invention, the polynucleotide construct comprises the Bs3₁₄ₓ super promoter operably linked to a nucleotide sequence encoding the execution protein AvrGf1. In another embodiment of the invention, the polynucleotide construct comprises the Bs3_{4X} short promoter operably linked to a nucleotide sequence encoding the execution protein AvrGf1.

Additionally provided are citrus plants, plant cells, and other host cells, isolated nucleic acid molecules, and expression comprising the polynucleotide constructs and promoters of the present invention.

### BRIEF DESCRIPTION OF THE DRAWINGS

FIG. 1. The Bs3 promoter (SEQ ID NO: 1). This sequence is the 360 bp upstream of ATG. The UPA box is shown in bold and underlined. The primer binding sites, which produce 200 bp fragment of the Bs3 promoter in a PCR amplification, are shown in italics. The UPA box that is targeted by AvrBs3

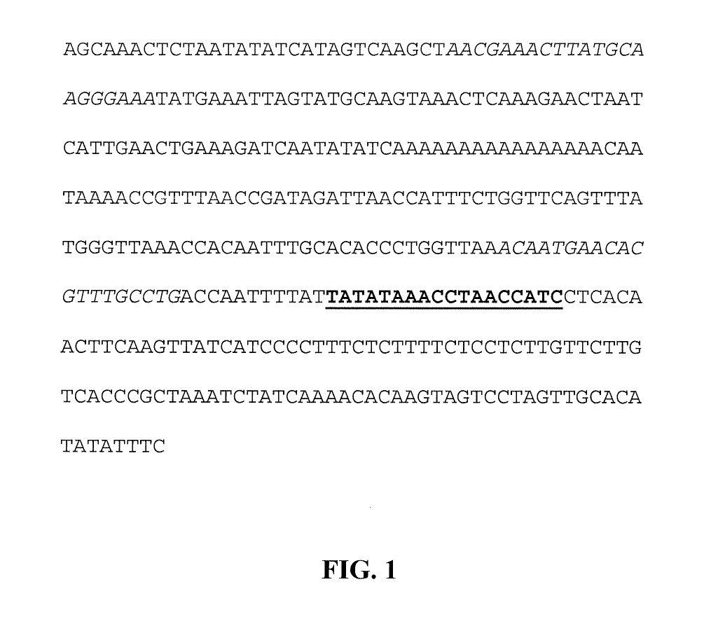

FIG. 2. The Bs3₁₄ₓ super promoter (SEQ ID NO: 2). Using site-directed mutagenesis AgeI (ACCGGT) and XhoI (CTCGAG) were introduced into the Bs3 promoter. The complex promoter was synthesized with flanking AgeI and XhoI recognition sites (boxed) and cloned into the Bs3 promoter. The synthesized fragment extends from the AgeI recognition site to the XhoI recognition site. The UPT boxes are shown in bold and underlined with name shown above each box. The UPT box that is targeted by AvrBs3 is part of the Bs3 wild-type promoter and is found outside of the synthesized region toward the 3′ end of the Bs3₁₄ₓ super promoter. The primer binding sites are shown in italics. The Bs3₁₄ₓ super promoter also referred to herein as the “Bs3 super promoter”.

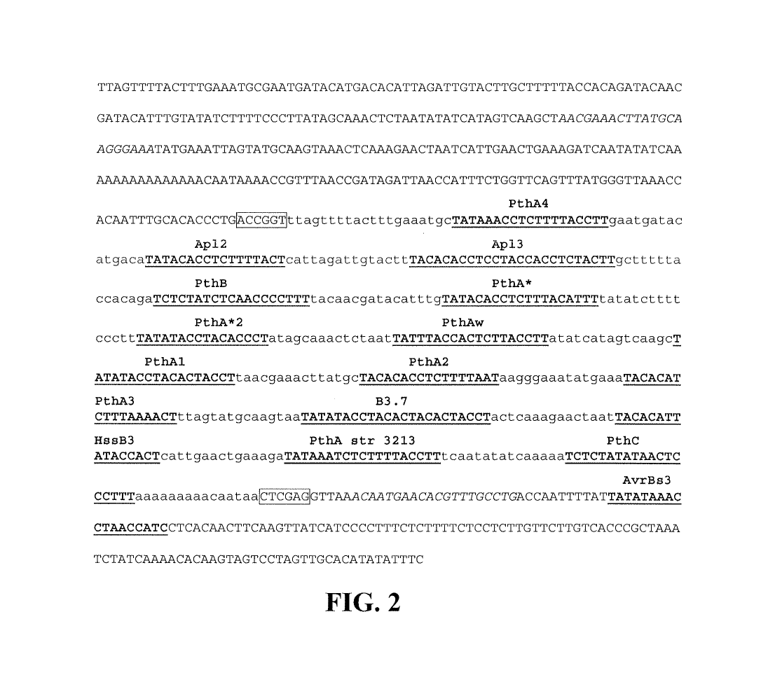

FIG. 3. The Bs3_{4X} short promoter (SEQ ID NO: 3). Based on the Bs3 promoter sequence, the additional UPT boxes are shown in bold with name shown above each box. To distinguish where one adjacent UPT box ends and the next begins, the first and third UPT boxes are underlined. The UPT boxes in the Bs3_{4X} short promoter are in order from the 5′ to 3′ direction: PthA4 strain 306 (underlined), B3.7 strain KC-21 (no underline, Apl2 strain NA-1 (underlined) and AvrTAw strain Aw (no underline).

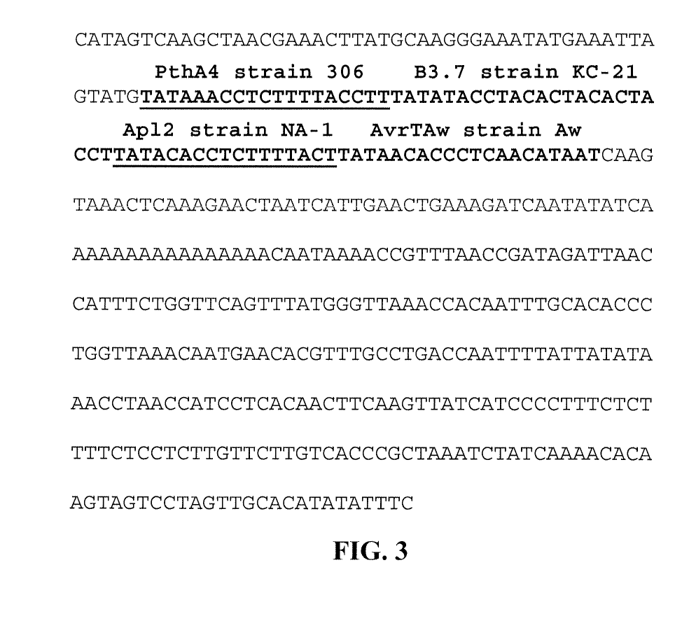

FIG. 4. The amino acid sequence of AvrGf1 (Accession No. ABB84189.1).

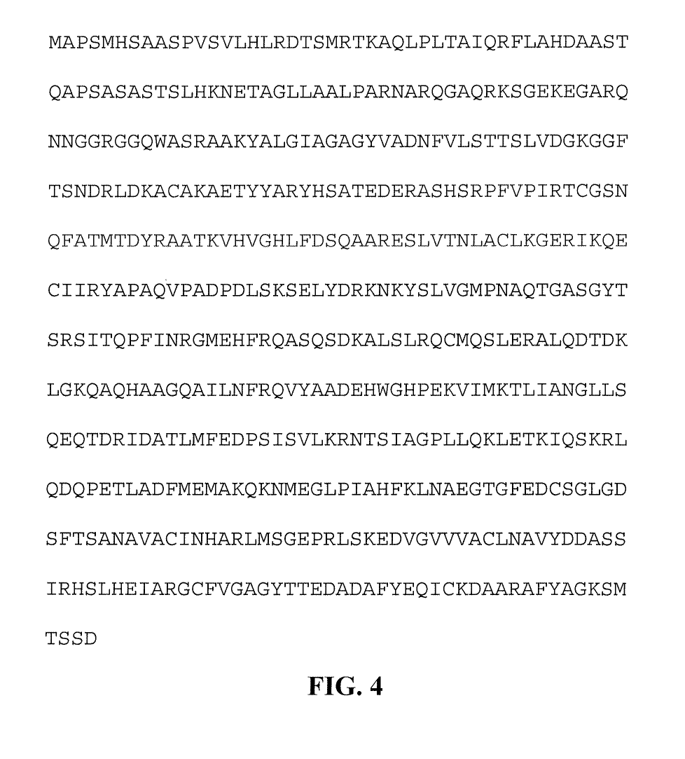

FIG. 5. Expression of avrGf1 in grapefruit leaf tissue is tightly regulated by the Bs3 promoter. FIG. 5A. Intact grapefruit leaves were transiently transformed with 31+Bs3::avrGf1 (avrGf1) strain and co-inoculated with Xcc-306 (right leaf) and Xcc-306+avrBs3 (left leaf); FIG. 5B. Same inoculations as in the panel A four days after inoculation; FIG. 5C. grapefruit leaves transiently transformed with 31+Bs3::avrGf1 (avrGf1) strain and co-inoculated with 306ΩhrpG⁻ mutant−hrp⁻ (right leaf) and 306ΩhrpG⁻ mutant+avrBs3 (left leaf).

FIG. 6. Bs3 promoter recognizes AvrHah1, an avrBs3 homolog from Xanthomonas gardneri. Grapefruit leaves were transiently transformed with 31+Bs3::avrGf1 (avrGf1) and co-inoculated with X. gardneri (avrHah1) and X. gardneri avrHah1⁻ mutant (avrHah1⁻). Left side: the strains were infiltrated alone without co-inoculations; Right side: 31+Bs3::avrGf1 strain was infiltrated and co-inoculated with, either, X. gardneri and X. gardneri avrHah1⁻ after five hours.

FIG. 7. In planta growth of X. citri strain 306 (Xcc-306); A. tumefaciens strain GV3101 co-inoculated with Xcc-306 (GV3101+Xcc-306); A. tumefaciens strain GV3101 containing Bs3::avrGf1 co-inoculated with Xcc-306 (31Bs3+Xcc-306); and 31+Bs3::avrGf1 co-inoculated with Xcc-306 containing pLAT211 (31Bs3+Xcc-306::avrBs3) in grapefruit leaves at different times after infiltration of 5×10⁸ cfu/mL of each strain into mesophyll.

FIG. 8. Comparison of GUS activity assay in grapefruit leaves transiently transformed with Agrobacterium strain GV3101 containing pK7Bs3::GUS (blue) and pK7Bs3₁₄ₓ::GUS (orange) constructs and co-inoculated with several X. citri strains. The infiltrated leaves were assessed five days after inoculation. The reading was taken 16 hours after incubation at 37° C. GUS activity is the average of three independent experiments.

FIG. 9. Comparison of GUS activity in grapefruit leaves after transient transformation with pK7Bs3::GUSi, pK7Bs3₄ₓ::GUSi, and pK7Bs3₁₄ₓ::GUSi constructs and co-inoculated with Xcc-306 and 306+avrBs3. The infiltrated leaves were assessed five days after inoculation. The reading was taken 16 hours after incubation at 37° C. GUS activity is the average of three independent experiments.

FIG. 10. Stably transformed grapefruit lines resistant to X. citri (A) Transgenic grapefruit transformed with Bs3 native promoter regulating the expression of Bs3 pepper gene (Bs3::Bs3cds). (B) Transgenic grapefruit transformed with Bs3 native promoter regulating the expression of the execution avrGf1 gene from X. citri strain A^{w} (Bs3::avrGf1). Both were infiltrated with X. citri strain 306 carrying the avrBs3 gene (Xcc-306+avrBs3). The pictures were taken 28 days after infiltration.

### SEQUENCES

The nucleotide and amino acid sequences listed in the accompanying sequence listing and/or drawings or otherwise provided herein are shown using standard letter abbreviations for nucleotide bases, and three-letter code for amino acids. The nucleotide sequences follow the standard convention of beginning at the 5′ end of the sequence and proceeding forward (i.e., from left to right in each line) to the 3′ end. Only one strand of each nucleic acid sequence is shown, but the complementary strand is understood to be included by any reference to the displayed strand. The amino acid sequences follow the standard convention of beginning at the amino terminus of the sequence and proceeding forward (i.e., from left to right in each line) to the carboxy terminus.

SEQ ID NO: 1 sets forth a nucleotide sequence comprising the Bs3 promoter.

SEQ ID NO: 2 sets forth the nucleotide sequence of the Bs3₁₄ₓ super promoter.

SEQ ID NO: 3 sets forth the nucleotide sequence of the Bs3_{4X} short promoter.

SEQ ID NO: 4 sets forth the amino acid sequence of the AvrGf1 (Accession No. ABB84189.1).

SEQ ID NO: 5 sets forth the nucleotide sequence of the UPT_{Apl1} box used in the Bs3₁₄ₓ super promoter comprising the nucleotide sequence set forth in SEQ ID NO: 2 and in the Bs3_{4X} short promoter comprising the nucleotide sequence set forth in SEQ ID NO: 3.

SEQ ID NO: 6 sets forth the nucleotide sequence of the UPT_{Apl2} box used in the Bs3₁₄ₓ super promoter comprising the nucleotide sequence set forth in SEQ ID NO: 2 and in the Bs3_{4X} short promoter comprising the nucleotide sequence set forth in SEQ ID NO: 3.

SEQ ID NO: 7 sets forth the nucleotide sequence of the UPT_{Apl3} box used in the Bs3₁₄ₓ super promoter comprising the nucleotide sequence set forth in SEQ ID NO: 2.

SEQ ID NO: 8 sets forth the nucleotide sequence of the UPT_{PthB} box used in the Bs3₁₄ₓ super promoter comprising the nucleotide sequence set forth in SEQ ID NO: 2.

SEQ ID NO: 9 sets forth the nucleotide sequence of the UPT_{pthA*} box used in the Bs3₁₄ₓ super promoter comprising the nucleotide sequence set forth in SEQ ID NO: 2.

SEQ ID NO: 10 sets forth the nucleotide sequence of the UPT_{pthA*2} box used in the Bs3₁₄ₓ super promoter comprising the nucleotide sequence set forth in SEQ ID NO: 2.

SEQ ID NO: 11 sets forth the nucleotide sequence of the UPT_{pthAw} box used in the Bs3₁₄ₓ super promoter comprising the nucleotide sequence set forth in SEQ ID NO: 2.

SEQ ID NO: 12 sets forth the nucleotide sequence of the UPT_{pthA1} box used in the Bs3₁₄ₓ super promoter comprising the nucleotide sequence set forth in SEQ ID NO: 2.

SEQ ID NO: 13 sets forth the nucleotide sequence of the UPT_{pthA2} box used in the Bs3₁₄ₓ super promoter comprising the nucleotide sequence set forth in SEQ ID NO: 2.

SEQ ID NO: 14 sets forth the nucleotide sequence of the UPT_{PthA3} box used in the Bs3₁₄ₓ super promoter comprising the nucleotide sequence set forth in SEQ ID NO: 2.

SEQ ID NO: 15 sets forth the nucleotide sequence of the UPT_{pB3.7} box used in the Bs3₁₄ₓ super promoter comprising the nucleotide sequence set forth in SEQ ID NO: 2 and in the Bs3_{4X} short promoter comprising the nucleotide sequence set forth in SEQ ID NO: 3.

SEQ ID NO: 16 sets forth the nucleotide sequence of the UPT_{HssB3.0} box used in the Bs3₁₄ₓ super promoter comprising the nucleotide sequence set forth in SEQ ID NO: 2.

SEQ ID NO: 17 sets forth the nucleotide sequence of the UPT_{PthA} box used in the Bs3₁₄ₓ super promoter comprising the nucleotide sequence set forth in SEQ ID NO: 2.

SEQ ID NO: 18 sets forth the nucleotide sequence of the UPT_{PthC} box used in the Bs3₁₄ₓ super promoter comprising the nucleotide sequence set forth in SEQ ID NO: 2.

SEQ ID NO: 19 sets forth the nucleotide sequence of the UPT_{AvrTAw} box used in the Bs3_{4X} short promoter comprising the nucleotide sequence set forth in SEQ ID NO: 3.

SEQ ID NOS: 20-34 set forth the amino acid sequences shown in Table 4. Each of the amino acid sequences in Table 4 comprises the consecutive repeat variable diresidues (RVDs) from the repeat domains of a TAL effector from a particular Xanthomonas strains. SEQ ID NOS: 20-34 do not set forth amino acid sequences that are known to occur in any of the TAL effectors of the various Xanthomonas strains in Table 4. Within a TAL effector, each RVD is separated from an adjacent RVD by multiple amino acids.

### DETAILED DESCRIPTION OF THE INVENTION

Recently, the pepper (Capsicum annuum) Bs3 resistance (R) gene was isolated, sequenced, and characterized. See, Römer et al. (2007) Science 318:645-648, U.S. Patent Application Publication No. 2009/0133158, and WO 2009/042753; all of which are herein incorporated in their entirety by reference. Molecular analysis revealed that the Bs3 promoter contains an element known as a UPA box and that the bacterial effector protein AvrBs3 binds to the UPA box and activates the Bs3 promoter. Additional chararcterization of the UPA box of the Bs3 promoter, related promoters, and synthetic promoters are disclosed in Römer et al. (2009) PNAS 106:20526-20531, U.S. Patent Application Publication No. 2010/0132069, and WO 2010/054348; all of which are herein incorporated in their entirety by reference.

The production of citrus has become imperiled by the unabated spread of ACC. The United States is the third largest citrus producer in the world, with the greatest citrus production occurring in Florida, valued at more than $9 billion (Boriss (2006) Commodity profile: Citrus Agriculture Marketing Resource Center, University of California; Hodges et al. (2006) Economic impacts of the Florida citrus industry in 2003-04, University of Florida, Institute for Food and Agriculture Sciences, EDIS document FE633). Severe economic consequences from citrus canker have occurred from the loss of marketability of fruit, reduction in fruit production and tree vigor, extra control measures, and the substantial cost incurred by eradication efforts. Various strains of Xanthomonas are known to cause citrus canker (Table 1). Unsuccessful attempts to eliminate the disease between 1996 and 2006 by eradication resulted in a cost of $1.2 billion and the destruction of 7 million commercial and 5 million nursery and residential trees (Bausher et al. (2006) BMC Plant Biol. 6:21), the largest plant-pest eradication effort ever carried out in the U.S. No new solutions have yet been deployed, and the recommended alternative management strategies are to plant windbreaks, minimize the establishment of disease with copper sprays, and control populations of leafminer, which contribute to disease spread (Graham et al. (2007) 2008 Florida citrus pest management guide for citrus canker, University of Florida, Institute for Food and Agriculture Sciences, EDIS document PP-182). These methods do limit the extent of disease; however they are inadequate to provide effective control, and they incur additional costs, have chemical safety issues and may not be durable (Canteros (2002) Phytopathol. 92:S116). The use of other chemical control measures, such as induced systemic resistance compounds, has also been ineffective (Graham et al. (2004). Mol. Plant Pathol. 5:1-15). The preferred control method for citrus canker, as indeed with all plant diseases, is genetic resistance, because it is generally more effective and environmentally benign. Therefore, new strategies for genetic resistance in citrus species are needed to combat the epidemic of citrus canker in Florida and other afflicted, citrus-growing regions of the world.

A non-limiting list of Xanthomonas strains that cause citrus canker is provided in Table 1.

TABLE 1
Xanthomonas Strains Causing Canker on Citrus

| Strain | | | |
|---|---|---|---|
| Designation | Pathovar name(s) | Geography | Species effected |
| A, Asiatic | Xanthomonas citri subsp. | Argentina, Bolivia, Brazil, China, | Wide range, high pathogenicity on |
| | citri | Florida, Hong Kong, India, Japan. | sweet orange, grapefruit, Key |
| | Also known as: | Malaysia, Mauritius, Pakistan, | Lime. Mandarin is more resistant. |
| | X. campestris pv citri | Paraguay, Philippines, Reunion Is, | |
| | Strain A | Rodrigues Is, Taiwan, Thailand, | |
| | X. axonopodis pv citri | Uruguay, Vietnam | |
| | X. smithii subsp citri | | |
| Aw | Same as A | Florida | Key Lime, other citrus are |
| | | | immune. |
| A* | Same as A | India, Iran, Saudi Arabia | Key Lime, other citrus are |
| | | | immune. |
| B, Cancrosis B | X. fuscans subsp. | Argentina, Uruguay | Key Lime, lemons. |
| | aurantifolii | | |
| C, Cancrosis C | X. fuscans subsp. | Brazil | Key Lime |
| | aurantifolii | | |

The present invention provides citrus plants with enhanced resistance to Asiatic citrus canker (ACC) and/or other citrus canker causing species and strains of Xanthomonas such as, for example, those strains and species listed in Table 1. Additionally provided are methods and compositions for making such citrus plants. Thus, the present invention finds use in combating the epidemic of ACC in Florida and other afflicted, citrus-growing regions of the world.

The present invention is based on the discovery that a polynucleotide construct comprising a promoter inducible by a Xanthomonas strain that causes ACC operably linked to an execution gene can cause a hypersensitive response (HR) in a citrus plant when a citrus plant comprising the polynucleotide construct is infected with the Xanthomonas strain. The execution gene of the present invention encodes the protein that can cause cell death when expressed in a plant or cell thereof. Such a protein is referred to herein as an execution protein. In one embodiment of the invention the execution protein is AvrGf1, which is encoded by the avrGf1 gene from X. citri strain A^{w}. The amino acid sequence of AvrGf1 is set forth in SEQ ID NO:4.

Certain embodiments of the invention are based on the further discovery that a Bs3 promoter can be engineered to contain multiple UPT boxes that each correspond to and can be induced by specific TAL effectors of Xanthomonas strains that cause citrus canker, particularly ACC, and moreover that such a promoter can be operably linked to an execution gene and used to produce citrus trees with resistance to multiple Xanthomonas strains that cause ACC and/or other forms of citrus canker caused by Xanthomonas strains. Thus, the present invention finds use in agriculture, particularly citrus production, by providing citrus trees with broad spectrum resistance to ACC and other forms of citrus canker caused by Xanthomonas strains.

The present invention provides methods for making a citrus plant with enhanced resistance to citrus canker, particularly Asiatic citrus canker (ACC). The methods of the present invention involve transforming at least one citrus plant cell a polynucleotide construct comprising a promoter operably linked to a coding sequence of an execution gene, wherein said promoter comprises at least one UPT box, and wherein said execution gene encodes an execution protein that is capable of triggering cell death in a citrus plant. The methods can further involve regenerating a transformed citrus plant from said citrus plant cell, wherein said transformed citrus plant comprises enhanced resistance to at least one Xanthomonas strain that causes citrus canker, particularly a Xanthomonas strain that causes ACC.

In a preferred embodiment, the present invention provides methods for making a citrus plant with enhanced resistance to ACC. The methods of the present invention involve transforming at least one citrus plant cell a polynucleotide construct comprising a promoter operably linked to a coding sequence of an execution gene, wherein said promoter comprises at least one UPT box, and wherein said execution gene encodes an execution protein that is capable of triggering cell death in a citrus plant. The methods can further involve regenerating a transformed citrus plant from said citrus plant cell, wherein said transformed citrus plant comprises enhanced resistance to at least one Xanthomonas strain that causes ACC.

For the present invention, “UPT box” is intended to mean a promoter element that specifically binds with an AvrBs3-like protein, also referred to as a TAL effector, and that a promoter comprising such a UPT box is capable, in the presence of its TAL effector, of inducing or increasing the expression of an operably linked nucleic acid molecule. “UPT boxes” are also referred to as “UPA boxes”, in particular the UPT box which is UP-regulated by AvrBs3, the first such UPT sequence to be characterized. Unless stated otherwise or readily apparent from the context, “UPT box” and “UPA box” as used herein are equivalent terms that can be used interchangeably and that do not differ in meaning and/or scope.

For many of the Xanthomonas strains that are known to cause ACC and other forms of citrus canker, the TAL effectors are known and include, but are not limited to, those set forth in Table 2. For many of these Xanthomonas strains, the UPT boxes are also known and are provided in Table 3. The repeat variable diresidues (RVDs) of TAL effector from various Xanthomonas strains and their corresponding UPT boxes for are provided in Table 4.

TABLE 2
Citrus TAL Effectors

| Strain, | | | | Protein | Protein | Highly related Pth |
|---|---|---|---|---|---|---|
| ID | Origin | Gene name¹ | Reference | database ID | size (aa) | proteins² |
| A, 3213 | FL | PthA | Yang and Gabriel | AAC43587.1 | 1163 | PthA4, Apl1, PthA- |
| | | | (1995) Mol. Plant- | | | KC21, PthA^{w}, PthA* |
| | | | Microbe Interact. | | | |
| | | | 8: 627-631 | | | |
| | | PthA1-3213 | Al-Saadi et al. | Not deposited | | |
| | | PthA2-3213 | (2007) Mol. Plant- | | | |
| | | PthA3-3213 | Microbe Interact, | | | |
| | | | 20: 934-943 | | | |
| A, 306 | Brazil | PthA1 | Da Silva et al. | AAM39226 | 1126 | PthA-KC21, PthA4, |
| | | | (2002) Nature | | | Apl1 |
| | | PthA2 | 417: 459-465 | AAM39243 | 1096 | B3.1, PthA3, Apl2 |
| | | PthA3 | | AAM39261 | 1096 | B3.1, PthA2, Apl2, |
| | | | | | | PthA*2 |
| | | PthA4 | | AAM39311.1 | 1163 | Apl1, PthA, PthA- |
| | | | | | | KC21, PthA^{w}, PthA* |
| A, NA-1 | Japan | Apl1 | Kanamori and | BAA37119 | 1163 | PthA4, PthA, PthA- |
| | | | Tsuyumu(1998) | | | KC21, PthA^{w}, PthA* |
| | | Apl2 | Annu. Phytopath. | BAA37120 | 1095 | PthA2, B3.1, PthA3 |
| | | Apl3 | Soc. Jpn 64: 462-470 | BAA37121 | 1367 | None |
| A, KC21 | Japan | PthA-KC21 | Shiotani et al. | BAF46271 | 1163 | PthA4, Apl1, PthA, |
| | | | (2007) J. Bacteriol. | | | PthA^{w}, PthA* |
| | | B3.7 | 189: 3271-3279 | BAF46272 | 1295 | None |
| | | B3.1 | | BAF46270 | 1096 | PthA3, PthA2, Apl2, |
| | | | | | | PthA*2 |
| | | HssB3.0 | | BAF46269 | 1060 | PthA3, B3.1, PthA2 |
| A^{w}, | FL | PthA^{w} | Al-Saadi et al. | ABO77779 | 1164 | Apl1, PthA4, PthA, |
| X0053 | | | (2007) Mol. Plant- | | | PthA- KC21, PthA* |
| | | | Microbe Interact. | | | |
| | | | 20: 934-943 | | | |
| | | Taw | Ryback et al. | ACN39605 | 795 | Apl3 |
| | | | (2009) Mol. Plant | | | |
| | | | Pathol. 10: 249-262 | | | |
| A*, | Saudi | PthA* | Al-Saadi et al. | ABO77780 | 1164 | PthA-KC21, PthA4, |
| Xc270 | Arabia | | (2007) Mol. Plant- | | | Apl1, PthA, Pth A^{w}, |
| | | PthA*2 | Microbe Interact. | ABO77781 | 1094 | B3.1, PthA3 |
| | | | 20: 934-943 | | | |
| B, B69 | Argentina | PthB | El Yacoubi et al. | AAO72098 | 1168 | PthC |
| | | B0 | (2007) Appl. | not deposited | | |
| | | | Environ. | | | |
| | | | Microbiol. | | | |
| | | | 73: 1612-1621 | | | |
| C, C340 | Brazil | PthC | Al-Saadi et al. | ABO77782 | 1168 | PthB |
| | | | (2007) Mol. Plant- | | | |
| | | | Microbe Interact. | | | |
| | | | 20: 934-943 | | | |

¹Primary TAL effectors are underlined.
²Pth homologs with >95% amino acid identity based on Blast scores of full-length proteins.

TABLE 3
UPT Boxes and Citrus Canker TAL Effectors

| | TAL | | | Accession |
|---|---|---|---|---|
| UPT Box | Effector | Species | Strain | No. |
| UPT_{Apl1} (SEQ ID NO: 5) | Apl1 | X. citri subsp. | A, Asiatic | NA-1 |
| TATAAACCTCTTTTACCTT | | citri | | |
| | PthA4 | X. citri subsp. | A, Asiatic | 306 |
| | | citri | | |
| | PthA-KC21 | X citri subsp. | A, Asiatic | KC21 |
| | | citri | | |
| UPT_{Apl2} (SEQ ID NO: 6) | Apl2 | X. citri subsp. | A, Asiatic | NA-1 |
| TATACACCTCTTTTACT | | citri | | |
| UPT_{Apl3} (SEQ ID NO: 7) | Apl3 | X. citri subsp. | A, Asiatic | NA-1 |
| TACACACCTCCTACCACCTCTACTT | | citri | | |
| UPT_{PthB} (SEQ ID NO: 8) | PthB | X. fuscans | B, Cancrosis B | B69 |
| TCTCTATCTCAACCCCTTT | | subsp. | | |
| | | aurantifoli | | |
| UPT_{PthA*} (SEQ ID NO: 9) | PthA* | X. citri subsp. | A* | Xc270 |
| TATACACCTCTTTACATTT | | citri | | |
| UPT_{PthA*2} (SEQ ID NO: 10) | PthA*2 | X. citri subsp. | A* | Xc270 |
| TATATACCTACACCCT | | citri | | |
| UPT_{PthAw} (SEQ ID NO: 11) | PthAw | X. citri subsp. | Aw | X0053 |
| TATTTACCACTCTTACCTT | | citri | | |
| UPT_{PthA1} (SEQ ID NO: 12) | PthA1 | X. citri subsp. | A, Asiatic | 306 |
| TATATACCTACACTACCT | | citri | | |
| UPT_{PthA2} (SEQ ID NO: 13) | PthA2 | X. citri subsp. | A, Asiatic | 306 |
| TACACACCTCTTTTAAT | | citri | | |
| UPT_{PthA3} (SEQ ID NO: 14) | PthA3 | X. citri subsp. | A, Asiatic | 306 |
| TACACATCTTTAAAACT | | citri | | |
| | pB3.1 | X. citri subsp. | A, Asiatic | KC21 |
| | | citri | | |
| UPT_{pB3.7} (SEQ ID NO: 15) | pB3.7 | X. citri subsp. | A, Asiatic | KC21 |
| TATATACCTACACTACACTACCT | | citri | | |
| UPT_{HssB3.0} (SEQ ID NO: 16) | HssB3.0 | X. dill subsp. | A, Asiatic | KC21 |
| TACACATTATACCACT | | citri | | |
| UPT_{PthA} (SEQ ID NO: 17) | PthA | X. citri subsp. | A, Asiatic | 3213 |
| TATAAATCTCTTTTACCTT | | citri | | |
| UPT_{PthC} (SEQ ID NO: 18) | PthC | X. fuscans subsp. | C, Cancrosis C | C340 |
| TCTCTATATAACTCCCTTT | | aurantifoli | | |

TABLE 4
TAL Effector Repeat Variable Diresidues (RVDs) for Various Xanthomonas
Strains and the Corresponding UPT Boxes

| Strain | TAL Effector RVDs/UPT Box¹ | SEQ ID NO | RD² |
|---|---|---|---|
| pthA 3213 | NINGNINININGHDNGHDNGNGNGNGNSHDHDNGNG | 20 | 17.5 |
| | A T A A A T C T C T T T T A C C T T | 17 | |
| pthA4 306 | NINGNININIHDHDNGHDNGNGNGNGNSHDHDNGNG | 21 | 17.5 |
| | A T A A A C C T C T T T T A C C T T | 5 | |
| apl1 NA1 | NINGNININIHDHDNGHDNGNGNGNGNSHDHDNGNG | 21 | 17.5 |
| | A T A A A C C T C T T T T A C C T T | 5 | |
| A-KC21 | NINGNININIHDHDNGHDNGNGNGNGNSHDHDNGNG | 21 | 17.5 |
| | A T A A A C C T C T T T T A C C T T | 5 | |
| PthAw | NINGNGNGNSHDHDNSHDNGNCNGNGNSHDHDNGNG | 22 | 17.5 |
| | A T T T A C C A C T C T T A C C T T | 11 | |
| PthA* | NINGNIHDNIHDHDNGHDNGNGNGNSHDNSNGNGNG | 23 | 17.5 |
| | A T A C A C C T C T T T T C A T T T | 9 | |
| B3.7 KC21 | NINGNINGNIHDHDNGNIHDNIHDNGNIHDNIHDNGNIHDHDNG | 24 | 21.5 |
| | A T A T A C C T A C A C T A C A C T A C C T | 15 | |
| PthA1 306 | NINGNINGNIHDHDNGNIHDNIHDNGNIHDHDNG | 25 | 16.5 |
| | A T A T A C C T A C A C T A C C T | 12 | |
| PthA*2 | NINGNINGNIHDHDNGNIHDNIHDHDHDNG | 26 | 14.5 |
| | A T A T A C C T A C A C C C T | 10 | |
| apl3 NA-1 | NIHDNIHDNIHDHDNGHDHDNGNIHDHDNIHDHDNGHDHGHIHDNGNG | 27 | 23.5 |
| | A C A C A c c T C C T A C C A C C T C T A C T T | 7 | |
| apl2 NA-1 | NINGNIHDNIHDHDNGHDNGNGNGNGNIHDNG | 28 | 15.5 |
| | A T A C A C C T C T T T T A C T | 6 | |
| PthA2 306 | NIHDNIHDNIHDHDNGHDNGNGNGNGNINING | 29 | 15.5 |
| | A C A C A C C T C T T T T A A T | 13 | |
| B3.1 KC21 | NIHDNIHDNINGHDNGNGNGNINININIHDNG | 30 | 15.5 |
| | A C A C A T C T T T A A A A C T | 14 | |
| PthA3 306 | NIHDNIHDNINGHDNGNGNGNINININIHDNG | 30 | 15.5 |
| | A C A C A T C T T T A A A A C T | 14 | |
| HssB3.0 KC21 | NIHDNIHDNINGNGNINGNIHDHDNIHDNG | 31 | 14.5 |
| | A C A C A T T A T A C C A C T | 16 | |
| PthB | HDNGHDNGNINGHDNGHDNINIHDHDHDHDNGNGNG | 32 | 17.5 |
| | C T C T A T C T C A A C C C C T T T | 8 | |
| PthC | HDNGHDHDNINGNINGNINIHDNGHDHDHDNGNGNG | 33 | 17.5 |
| | C T C C A T A T A A C T C C C T T T | 18 | |
| AvrTAw | NINGNINIHDNIHDHDHDNGHDNSNIHDNINGNINSNG | 34 | 18.5 |
| | A T A A C A C C C T C A A C A T A A T | 19 | |

¹The 5′-T was omitted from each UPT box because the5′-T does not have a corresponding RVD.
²Number of repeat domains (RD) in TAL effector.

Preferably, a promoter of the present invention comprises at least one UPT box that is capable of binding with at least one TAL effector from at least one Xanthomonas strain that is known to cause citrus canker. More preferably, the promoter comprises 2, 3, 4, 5, 6, 7, 8, 9, 10, 11, 12, 13, 14, 15 or more different UPT boxes and thus, is inducible by 2, 3, 4, 5, 6, 7, 8, 9, 10, 11, 12, 13, 14, 15 or more TAL effectors naturally occurring in Xanthomonas strains that are known to cause citrus canker. Preferred promoters of the present invention include the Bs3 promoter comprising the nucleotide sequence set forth in SEQ ID NO: 1, the Bs3₁₄ₓ super promoter comprising the nucleotide sequence set forth in SEQ ID NO: 2, the Bs3_{4X} short promoter comprising the nucleotide sequence set forth in SEQ ID NO: 3.

The promoters of the present invention can be operably linked to an execution gene of the present invention. Such execution genes encode proteins that are capable of causing cell death that it typically associated with a hypersensitive response when the protein is present in a plant cell, particularly a citrus plant cell. In one embodiment of the invention the execution gene comprises a nucleotide sequence encoding AvrGf1. The amino acid sequence of AvrGf1 is provided in SEQ ID NO: 4.

The methods of the present invention can be used with any citrus species that is susceptible to citrus canker caused by Xanthomonas. Citrus species of interest are those citrus species that are grown commercially. Such citrus species include, but are not limited to, grapefruit (Citrus×paradise), sweet orange (Citrus×sinensis), lemon (Citrus×limon), and Key lime (Citrus aurantifolia).

The invention encompasses isolated or substantially purified polynucleotide (also referred to herein as “nucleic acid molecules”) or protein (also referred to herein as “polypeptide”) compositions. An “isolated” or “purified” polynucleotide or protein, or biologically active portion thereof, is substantially or essentially free from components that normally accompany or interact with the polynucleotide or protein as found in its naturally occurring environment. Thus, an isolated or purified polynucleotide or protein is substantially free of other cellular material or culture medium when produced by recombinant techniques, or substantially free of chemical precursors or other chemicals when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For example, in various embodiments, the isolated polynucleotide can contain less than about 5 kb, 4 kb, 3 kb, 2 kb, 1 kb, 0.5 kb, or 0.1 kb of nucleotide sequence that naturally flank the polynucleotide in genomic DNA of the cell from which the polynucleotide is derived. A protein that is substantially free of cellular material includes preparations of protein having less than about 30%, 20%, 10%, 5%, or 1% (by dry weight) of contaminating protein. When the protein of the invention or biologically active portion thereof is recombinantly produced, optimally culture medium represents less than about 30%, 20%, 10%, 5%, or 1% (by dry weight) of chemical precursors or non-protein-of-interest chemicals.

Fragments and variants of the disclosed polynucleotides and proteins encoded thereby are also encompassed by the present invention. By “fragment” is intended a portion of the polynucleotide or a portion of the amino acid sequence and hence protein encoded thereby. Fragments of polynucleotides comprising coding sequences may encode protein fragments that retain biological activity of the native protein. Fragments of polynucleotide comprising promoter sequences retain biological activity of the full-length promoter, particularly promoter activity. Alternatively, fragments of a polynucleotide that are useful as hybridization probes generally do not encode proteins that retain biological activity or do not retain promoter activity. Thus, fragments of a nucleotide sequence may range from at least about 20 nucleotides, about 50 nucleotides, about 100 nucleotides, and up to the full-length polynucleotide of the invention.

A fragment of a polynucleotide of the invention may encode a biologically active portion of a promoter. A biologically active portion of a promoter of the present invention can be prepared by isolating a portion of one of the polynucleotides of the invention that comprises the promoter as described herein. Polynucleotides that are fragments of a nucleotide sequence of the present invention comprise at least 9, 10, 11, 12, 13, 14, 15, 16, 17, 18, 19, 20, 21, 22, 23, 24, 25, 30, 35, 40, 45, 50, 75, 100, 125, 150, 175, 200, 250, 300, 350, 400, 450, 500, 550, 600, 650, 700, 800, 900, 1000, 1100, 1200, 1300, 1400, 1500, 2000, 2500, or 3000 contiguous nucleotides, or up to the number of nucleotides present in a full-length polynucleotide disclosed herein.

“Variants” is intended to mean substantially similar sequences. For polynucleotides, a variant comprises a polynucleotide having deletions (i.e., truncations) at the 5′ and/or 3′ end; deletion and/or addition of one or more nucleotides at one or more internal sites in the native polynucleotide; and/or substitution of one or more nucleotides at one or more sites in the native polynucleotide. As used herein, a “native” polynucleotide or polypeptide comprises a naturally occurring nucleotide sequence or amino acid sequence, respectively. For polynucleotides that comprise coding sequences, conservative variants include those sequences that, because of the degeneracy of the genetic code, encode the amino acid sequence of one of the polypeptides of the invention. Naturally occurring allelic variants such as these can be identified with the use of well-known molecular biology techniques, as, for example, with polymerase chain reaction (PCR) and hybridization techniques as outlined below. Variant polynucleotides also include synthetically derived polynucleotides, such as those generated, for example, by using site-directed mutagenesis but which still comprise promoter activity. Generally, variants of a particular polynucleotide or nucleic acid molecule of the invention will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that particular polynucleotide as determined by sequence alignment programs and parameters as described elsewhere herein.

Preferred fragments and variants of a promoter of the present invention comprise the promoter activity of the native promoter. One skilled in the art will appreciate that such fragments and variants of a promoter be evaluated by routine screening assays such as, for example, the transient promoter activity assays described hereinbelow, wherein the promoter is operably linked to a nucleotide sequence encoding AvrGf1 or GUS (β-glucoronidase). Such transient assays can be used to evaluate the activity of individual fragments and variants of the Bs3₁₄ₓ super promoter and the Bs3_{4X} short promoter.

Preferred fragments and variants of a Bs3₁₄ₓ super promoter comprise Bs3₁₄ₓ super promoter activity. That is such fragments and variants of a Bs3₁₄ₓ super promoter are inducible by the same TAL effectors as the Bs3₁₄ₓ super promoter and in preferred embodiments, comprise promoter activity in plant or cell thereof that is the same or substantially the same as the Bs3₁₄ₓ super promoter.

Preferred fragments and variants of a Bs3_{4X} short promoter comprise Bs3_{4X} short promoter. That is such fragments and variants of a Bs3_{4X} short promoter are inducible by the same TAL effectors as the Bs3_{4X} short promoter and in preferred embodiments, comprise promoter activity in plant or cell thereof that is the same or substantially the same as the Bs3_{4X} short promoter.

Variants of a particular polynucleotide of the invention (i.e., the reference polynucleotide) can also be evaluated by comparison of the percent sequence identity between the polypeptide encoded by a variant polynucleotide and the polypeptide encoded by the reference polynucleotide. Percent sequence identity between any two polypeptides can be calculated using sequence alignment programs and parameters described elsewhere herein. Where any given pair of polynucleotides of the invention is evaluated by comparison of the percent sequence identity shared by the two polypeptides they encode, the percent sequence identity between the two encoded polypeptides is at least about 60%, 65%, 70%, 75%, 80%, 85%, 90%, 91%, 92%, 93%, 94%, 95%, 96%, 97%, 98%, 99% or more sequence identity.

“Variant” protein is intended to mean a protein derived from the native protein by deletion (so-called truncation) of one or more amino acids at the N-terminal and/or C-terminal end of the native protein; deletion and/or addition of one or more amino acids at one or more internal sites in the native protein; or substitution of one or more amino acids at one or more sites in the native protein. Variant proteins encompassed by the present invention are biologically active; that is they continue to possess the desired biological activity of the native protein. Such variants may result from, for example, genetic polymorphism or from human manipulation. Biologically active variants of a protein of the invention will have at least about 60%, 65%, 70%, 75%, 80%, 85%, 90%, 91%, 92%, 93%, 94%, 95%, 96%, 97%, 98%, 99% or more sequence identity to the amino acid sequence for the native protein as determined by sequence alignment programs and parameters described elsewhere herein. A biologically active variant of a protein of the invention may differ from that protein by as few as 1-15 amino acid residues, as few as 1-10, such as 6-10, as few as 5, as few as 4, 3, 2, or even 1 amino acid residue.

The proteins of the invention may be altered in various ways including amino acid substitutions, deletions, truncations, and insertions. Methods for such manipulations are generally known in the art. For example, amino acid sequence variants and fragments of the proteins can be prepared by mutations in the DNA. Methods for mutagenesis and polynucleotide alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington, D.C.), herein incorporated by reference. Conservative substitutions, such as exchanging one amino acid with another having similar properties, may be optimal.

Thus, the genes and polynucleotides of the invention include both the naturally occurring sequences as well as mutant forms. Likewise, the proteins of the invention encompass naturally occurring proteins as well as variations and modified forms thereof.

Such variants will continue to possess the desired biological activity. Obviously, the mutations that will be made in the DNA encoding the variant must not place the sequence out of reading frame and optimally will not create complementary regions that could produce secondary mRNA structure. See, EP Patent Application Publication No. 75,444.

The deletions, insertions, and substitutions of the protein sequences encompassed herein are not expected to produce radical changes in the characteristics of the protein. However, when it is difficult to predict the exact effect of the substitution, deletion, or insertion in advance of doing so, one skilled in the art will appreciate that the effect will be evaluated by routine screening assays. That is, the activity of an execution protein be can be evaluated by the transient assays as described herein below. For example, a nucleotide sequence encoding an execution protein or fragment or variant thereof can be operably linked to a promoter of the present invention or a constitutive promoter such as the CaMV 35 promoter and evaluated in a transient assay for HR as described herein below. Those fragments and variants of an execution protein will retain the ability of the execution protein to trigger HR when in plant or cell thereof. Fragments and variants of AvrGf1 retain the ability of AvrGf1 to trigger HR when in a plant or cell thereof as described herein. Such fragments and variants are referred to herein as comprising AvrGf1 activity.

Variant polynucleotides and proteins also encompass sequences and proteins derived from a mutagenic and recombinogenic procedure such as DNA shuffling. Strategies for such DNA shuffling are known in the art. See, for example, Stemmer (1994) Proc. Natl. Acad. Sci. USA 91:10747-10751; Stemmer (1994) Nature 370:389-391; Crameri et al. (1997) Nature Biotech. 15:436-438; Moore et al. (1997) J. Mol. Biol. 272:336-347; Zhang et al. (1997) Proc. Natl. Acad. Sci. USA 94:4504-4509; Crameri et al. (1998) Nature 391:288-291; and U.S. Pat. Nos. 5,605,793 and 5,837,458.

The polynucleotides of the invention can be used to isolate corresponding sequences from other organisms, particularly other plants. In this manner, methods such as PCR, hybridization, and the like can be used to identify such sequences based on their sequence homology to the sequences set forth herein. Sequences isolated based on their sequence identity to the entire sequences set forth herein or to variants and fragments thereof are encompassed by the present invention. Such sequences include sequences that are orthologs of the disclosed sequences. “Orthologs” is intended to mean genes derived from a common ancestral gene and which are found in different species as a result of speciation. Genes found in different species are considered orthologs when their nucleotide sequences and/or their encoded protein sequences share at least 60%, 70%, 75%, 80%, 85%, 90%, 91%, 92%, 93%, 94%, 95%, 96%, 97%, 98%, 99%, or greater sequence identity. Functions of orthologs are often highly conserved among species. Thus, isolated polynucleotides that have promoter activity and which hybridize under stringent conditions to at least one of the polynucleotides disclosed herein, or to variants or fragments thereof, are encompassed by the present invention.

In a PCR approach, oligonucleotide primers can be designed for use in PCR reactions to amplify corresponding DNA sequences from cDNA or genomic DNA extracted from any plant of interest. Methods for designing PCR primers and PCR cloning are generally known in the art and are disclosed in Sambrook et al. (1989) Molecular Cloning: A Laboratory Manual (2d ed., Cold Spring Harbor Laboratory Press, Plainview, N.Y.). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York). Known methods of PCR include, but are not limited to, methods using paired primers, nested primers, single specific primers, degenerate primers, gene-specific primers, vector-specific primers, partially-mismatched primers, and the like.

In hybridization techniques, all or part of a known polynucleotide is used as a probe that selectively hybridizes to other corresponding polynucleotides present in a population of cloned genomic DNA fragments or cDNA fragments (i.e., genomic or cDNA libraries) from a chosen organism. The hybridization probes may be genomic DNA fragments, cDNA fragments, RNA fragments, or other oligonucleotides, and may be labeled with a detectable group such as ³²P, or any other detectable marker. Thus, for example, probes for hybridization can be made by labeling synthetic oligonucleotides based on the polynucleotides of the invention. Methods for preparation of probes for hybridization and for construction of cDNA and genomic libraries are generally known in the art and are disclosed in Sambrook et al. (1989) Molecular Cloning: A Laboratory Manual (2d ed., Cold Spring Harbor Laboratory Press, Plainview, N.Y.).

For example, an entire nucleic acid molecule of polynucleotide disclosed herein, or one or more portions thereof, may be used as a probe capable of specifically hybridizing to corresponding polynucleotide and messenger RNAs. To achieve specific hybridization under a variety of conditions, such probes include sequences that are unique among one or more of the polynucleotide sequences of the present invention and are optimally at least about 10 nucleotides in length, and most optimally at least about 20 nucleotides in length. Such probes may be used to amplify corresponding polynucleotides from a chosen plant by PCR. This technique may be used to isolate additional coding sequences from a desired plant or as a diagnostic assay to determine the presence of coding sequences in a plant. Hybridization techniques include hybridization screening of plated DNA libraries (either plaques or colonies; see, for example, Sambrook et al. (1989) Molecular Cloning: A Laboratory Manual (2d ed., Cold Spring Harbor Laboratory Press, Plainview, N.Y.).

Hybridization of such sequences may be carried out under stringent conditions.

By “stringent conditions” or “stringent hybridization conditions” is intended conditions under which a probe will hybridize to its target sequence to a detectably greater degree than to other sequences (e.g., at least 2-fold over background). Stringent conditions are sequence-dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length, optimally less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C. Optionally, wash buffers may comprise about 0.1% to about 1% SDS. Duration of hybridization is generally less than about 24 hours, usually about 4 to about 12 hours. The duration of the wash time will be at least a length of time sufficient to reach equilibrium.

Specificity is typically the function of post-hybridization washes, the critical factors being the ionic strength and temperature of the final wash solution. For DNA-DNA hybrids, the Tₘ can be approximated from the equation of Meinkoth and Wahl (1984) Anal. Biochem. 138:267-284: Tₘ=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization, and/or wash conditions can be adjusted to hybridize to sequences of the desired identity. For example, if sequences with ≧90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution), it is optimal to increase the SSC concentration so that a higher temperature can be used. An extensive guide to the hybridization of nucleic acids is found in Tijssen (1993) Laboratory Techniques in Biochemistry and Molecular Biology—Hybridization with Nucleic Acid Probes, Part I, Chapter 2 (Elsevier, New York); and Ausubel et al., eds. (1995) Current Protocols in Molecular Biology, Chapter 2 (Greene Publishing and Wiley-Interscience, New York). See Sambrook et al. (1989) Molecular Cloning: A Laboratory Manual (2d ed., Cold Spring Harbor Laboratory Press, Plainview, N.Y.).

It is recognized that the polynucleotide molecules of the present invention encompass polynucleotide molecules comprising a nucleotide sequence that is sufficiently identical to one of the nucleotide sequences set forth in SEQ ID NOS: 6, 7, 9, 11, 13-18, 20, 22, or 24. The term “sufficiently identical” is used herein to refer to a first amino acid or nucleotide sequence that contains a sufficient or minimum number of identical or equivalent nucleotides to a second nucleotide sequence such that the first and second nucleotide sequences have a common structural domain and/or common functional activity. For example, nucleotide sequences that contain a common structural domain having at least about 45%, 55%, or 65% identity, preferably 75% identity, more preferably 85%, 90%, 95%, 96%, 97%, 98% or 99% identity are defined herein as sufficiently identical.

To determine the percent identity of two amino acid sequences or of two nucleic acids, the sequences are aligned for optimal comparison purposes. The percent identity between the two sequences is a function of the number of identical positions shared by the sequences (i.e., percent identity=number of identical positions/total number of positions (e.g., overlapping positions)×100). In one embodiment, the two sequences are the same length. The percent identity between two sequences can be determined using techniques similar to those described below, with or without allowing gaps. In calculating percent identity, typically exact matches are counted.

The determination of percent identity between two sequences can be accomplished using a mathematical algorithm. A preferred, nonlimiting example of a mathematical algorithm utilized for the comparison of two sequences is the algorithm of Karlin and Altschul (1990) Proc. Natl. Acad. Sci. USA 87:2264, modified as in Karlin and Altschul (1993) Proc. Natl. Acad. Sci. USA 90:5873-5877. Such an algorithm is incorporated into the NBLAST and XBLAST programs of Altschul et al. (1990) J. Mol. Biol. 215:403. BLAST nucleotide searches can be performed with the NBLAST program, score=100, wordlength=12, to obtain nucleotide sequences homologous to the polynucleotide molecules of the invention. BLAST protein searches can be performed with the XBLAST program, score=50, wordlength=3, to obtain amino acid sequences homologous to protein molecules of the invention. To obtain gapped alignments for comparison purposes, Gapped BLAST can be utilized as described in Altschul et al. (1997) Nucleic Acids Res. 25:3389. Alternatively, PSI-Blast can be used to perform an iterated search that detects distant relationships between molecules. See Altschul et al. (1997) supra. When utilizing BLAST, Gapped BLAST, and PSI-Blast programs, the default parameters of the respective programs (e.g., XBLAST and NBLAST) can be used. See http://www.ncbi.nlm.nih.gov. Another preferred, non-limiting example of a mathematical algorithm utilized for the comparison of sequences is the algorithm of Myers and Miller (1988) CABIOS 4:11-17. Such an algorithm is incorporated into the ALIGN program (version 2.0), which is part of the GCG sequence alignment software package. When utilizing the ALIGN program for comparing amino acid sequences, a PAM120 weight residue table, a gap length penalty of 12, and a gap penalty of 4 can be used. Alignment may also be performed manually by inspection.

Unless otherwise stated, sequence identity/similarity values provided herein refer to the value obtained using the full-length sequences of the invention and using multiple alignment by mean of the algorithm Clustal W (Nucleic Acid Research, 22(22):4673-4680, 1994) using the program AlignX included in the software package Vector NTI Suite Version 7 (InforMax, Inc., Bethesda, Md., USA) using the default parameters; or any equivalent program thereof. By “equivalent program” is intended any sequence comparison program that, for any two sequences in question, generates an alignment having identical nucleotide or amino acid residue matches and an identical percent sequence identity when compared to the corresponding alignment generated by CLUSTALW (Version 1.83) using default parameters (available at the European Bioinformatics Institute website: www.ebi.ac.uk/Tools/clustalw/index).

The use of the term “polynucleotide” is not intended to limit the present invention to polynucleotides comprising DNA. Those of ordinary skill in the art will recognize that polynucleotides, can comprise ribonucleotides and combinations of ribonucleotides and deoxyribonucleotides. Such deoxyribonucleotides and ribonucleotides include both naturally occurring molecules and synthetic analogues. The polynucleotides of the invention also encompass all forms of sequences including, but not limited to, single-stranded forms, double-stranded forms, hairpins, stem-and-loop structures, and the like.

The promoters of the present invention can be provided in expression cassettes for expression in the plant or other organism or host cell of interest. The cassette will include 5′ and 3′ regulatory sequences operably linked to polynucleotide to be expressed. “Operably linked” is intended to mean a functional linkage between two or more elements. For example, an operable linkage between a polynucleotide or gene of interest and a regulatory sequence (i.e., a promoter) is functional link that allows for expression of the polynucleotide of interest. Operably linked elements may be contiguous or non-contiguous. When used to refer to the joining of two protein coding regions, by operably linked is intended that the coding regions are in the same reading frame. The cassette may additionally contain at least one additional gene to be cotransformed into the organism. Alternatively, the additional gene(s) can be provided on multiple expression cassettes. Such an expression cassette is provided with a plurality of restriction sites and/or recombination sites for insertion of the polynucleotide to be under the transcriptional regulation of the regulatory regions. The expression cassette may additionally contain selectable marker genes.

The expression cassette will include in the 5′-3′ direction of transcription, a transcriptional and translational initiation region (i.e., a promoter), polynucleotide to be expressed, and a transcriptional and translational termination region (i.e., termination region) functional in plants or other organism or host cell. The regulatory regions (i.e., promoters, transcriptional regulatory regions, and translational termination regions) and/or the polynucleotide to be expressed may be native/analogous to the host cell or to each other. Alternatively, any of the regulatory regions and/or the polynucleotide to be expressed may be heterologous to the host cell or to each other. As used herein, “heterologous” in reference to a sequence is a sequence that originates from a foreign species, or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide. As used herein, a chimeric gene comprises a coding sequence operably linked to a transcription initiation region that is heterologous to the coding sequence.

The termination region may be native with the transcriptional initiation region, may be native with the operably linked polynucleotide of interest, may be native with the plant host, or may be derived from another source (i.e., foreign or heterologous) to the promoter, the polynucleotide of interest, the plant host, or any combination thereof. Convenient termination regions are available from the Ti-plasmid of A. tumefaciens, such as the octopine synthase and nopaline synthase termination regions. See also, Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acids Res. 15:9627-9639.

Unless stated otherwise or obvious from the context, a promoter of the present invention comprises a nucleotide sequence comprising at least one UPT box and is capable of directing the expression of an operably linked polynucleotide in a plant, a plant part, and/or a plant cell. Preferably, a promoter of the present invention is inducible in plants, particularly a citrus plant, by at least one Xanthomonas strain that is known to cause ACC. More preferably, the promoter is inducible by at least one Xanthomonas strain that is known to cause ACC and that produces a TAL effector. Most preferably, the promoter is inducible by at least one Xanthomonas strain that is known to cause ACC and that produces a TAL effector that specifically binds to at least one UPT box of the promoter.

Where appropriate, the polynucleotides may be optimized for increased expression in the transformed plant. That is, the polynucleotides can be synthesized using plant-preferred codons for improved expression. See, for example, Campbell and Gowri (1990) Plant Physiol. 92:1-11 for a discussion of host-preferred codon usage. Methods are available in the art for synthesizing plant-preferred genes. See, for example, U.S. Pat. Nos. 5,380,831, and 5,436,391, and Murray et al. (1989) Nucleic Acids Res. 17:477-498, herein incorporated by reference.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

The expression cassettes may additionally contain 5′ leader sequences. Such leader sequences can act to enhance translation. Translation leaders are known in the art and include: picornavirus leaders, for example, EMCV leader (Encephalomyocarditis 5′ noncoding region) (Elroy-Stein et al. (1989) Proc. Natl. Acad. Sci. USA 86:6126-6130); potyvirus leaders, for example, TEV leader (Tobacco Etch Virus) (Gallie et al. (1995) Gene 165(2):233-238), MDMV leader (Maize Dwarf Mosaic Virus) (Virology 154:9-20), and human immunoglobulin heavy-chain binding protein (BiP) (Macejak et al. (1991) Nature 353:90-94); untranslated leader from the coat protein mRNA of alfalfa mosaic virus (AMV RNA 4) (Jobling et al. (1987) Nature 325:622-625); tobacco mosaic virus leader (TMV) (Gallie et al. (1989) in Molecular Biology of RNA, ed. Cech (Liss, New York), pp. 237-256); and maize chlorotic mottle virus leader (MCMV) (Lommel et al. (1991) Virology 81:382-385). See also, Della-Cioppa et al. (1987) Plant Physiol. 84:965-968.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

The expression cassette can also comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones, and 2,4-dichlorophenoxyacetate (2,4-D). Additional selectable markers include phenotypic markers such as β-galactosidase and fluorescent proteins such as green fluorescent protein (GFP) (Su et al. (2004) Biotechnol Bioeng 85:610-9 and Fetter et al. (2004) Plant Cell 16:215-28), cyan florescent protein (CYP) (Bolte et al. (2004) J. Cell Science 117:943-54 and Kato et al. (2002) Plant Physiol 129:913-42), and yellow florescent protein (PhiYFP™ from Evrogen, see, Bolte et al. (2004) J Cell Science 117:943-54). For additional selectable markers, see generally, Yarranton (1992) Curr. Opin. Biotech. 3:506-511; Christopherson et al. (1992) Proc. Natl. Acad. Sci. USA 89:6314-6318; Yao et al. (1992) Cell 71:63-72; Reznikoff (1992) Mol. Microbiol. 6:2419-2422; Barkley et al. (1980) in The Operon, pp. 177-220; Hu et al. (1987) Cell 48:555-566; Brown et al. (1987) Cell 49:603-612; Figge et al. (1988) Cell 52:713-722; Deuschle et al. (1989) Proc. Natl. Acad. USA 86:5400-5404; Fuerst et al. (1989) Proc. Natl. Acad. Sci. USA 86:2549-2553; Deuschle et al. (1990) Science 248:480-483; Gossen (1993) Ph.D. Thesis, University of Heidelberg; Reines et al. (1993) Proc. Natl. Acad. Sci. USA 90:1917-1921; Labow et al. (1990) Mol. Cell. Biol. 10:3343-3356; Zambretti et al. (1992) Proc. Natl. Acad. Sci. USA 89:3952-3956; Baim et al. (1991) Proc. Natl. Acad. Sci. USA 88:5072-5076; Wyborski et al. (1991) Nucleic Acids Res. 19:4647-4653; Hillenand-Wissman (1989) Topics Mol. Struc. Biol. 10:143-162; Degenkolb et al. (1991) Antimicrob. Agents Chemother. 35:1591-1595; Kleinschnidt et al. (1988) Biochemistry 27:1094-1104; Bonin (1993) Ph.D. Thesis, University of Heidelberg; Gossen et al. (1992) Proc. Natl. Acad. Sci. USA 89:5547-5551; Oliva et al. (1992) Antimicrob. Agents Chemother. 36:913-919; Hlavka et al. (1985) Handbook of Experimental Pharmacology, Vol. 78 (Springer-Verlag, Berlin); Gill et al. (1988) Nature 334:721-724. Such disclosures are herein incorporated by reference.

The above list of selectable marker genes is not meant to be limiting. Any selectable marker gene can be used in the present invention.

Numerous plant transformation vectors and methods for transforming plants are available. See, for example, An, G. et al. (1986) Plant Pysiol., 81:301-305; Fry, J., et al. (1987) Plant Cell Rep. 6:321-325; Block, M. (1988) Theor. Appl Genet. 76:767-774; Hinchee, et al. (1990) Stadler. Genet. Symp. 203212.203-212; Cousins, et al. (1991) Aust. J Plant Physiol. 18:481-494; Chee, P. P. and Slightom, J. L. (1992) Gene. 118:255-260; Christou, et al. (1992) Trends. Biotechnol. 10:239-246; D'Halluin, et al. (1992) Bio/Technol. 10:309-314; Dhir, et al. (1992) Plant Physiol. 99:81-88; Casas et al. (1993) Proc. Nat. Acad Sci. USA 90:11212-11216; Christou, P. (1993) In Vitro Cell. Dev. Biol.-Plant; 29P:119-124; Davies, et al. (1993) Plant Cell Rep. 12:180-183; Dong, J. A. and Mchughen, A. (1993) Plant Sci. 91:139-148; Franklin, C. I. and Trieu, T. N. (1993) Plant. Physiol. 102:167; Golovkin, et al. (1993) Plant Sci. 90:41-52; Guo Chin Sci. Bull. 38:2072-2078; Asano, et al. (1994) Plant Cell Rep. 13; Ayeres N. M. and Park, W. D. (1994) Crit. Rev. Plant. Sci. 13:219-239; Barcelo, et al. (1994) Plant. J. 5:583-592; Becker, et al. (1994) Plant. J 5:299-307; Borkowska et al. (1994) Acta. Physiol Plant. 16:225-230; Christou, P. (1994) Agro. Food. Ind. Hi Tech. 5: 17-27; Eapen et al. (1994) Plant Cell Rep. 13:582-586; Hartman, et al. (1994) Bio-Technology 12: 919923; Ritala, et al. (1994) Plant. Mol. Biol. 24:317-325; and Wan, Y. C. and Lemaux, P. G. (1994) Plant Physiol. 104:3748.

The methods of the invention involve introducing a polynucleotide construct into a plant. By “introducing” what is intended is presenting to the plant the polynucleotide construct in such a manner that the construct gains access to the interior of a cell of the plant. The methods of the invention do not depend on a particular method for introducing a polynucleotide construct to a plant, only that the polynucleotide construct gains access to the interior of at least one cell of the plant. Methods for introducing polynucleotide constructs into plants are known in the art including, but not limited to, stable transformation methods, transient transformation methods, and virus-mediated methods.

By “stable transformation” is intended that the polynucleotide construct introduced into a plant integrates into the genome of the plant and is capable of being inherited by progeny thereof. By “transient transformation” is intended that a polynucleotide construct introduced into a plant does not integrate into the genome of the plant.

Certain embodiments of the methods of the invention involve stably transforming a plant or cell thereof with a polynucleotide construct comprising a promoter operably linked to a coding sequence of an execution gene. The present invention is not limited to introducing the polynucleotide construct into the plant or plant cell as a single nucleic acid molecule but also includes, for example, introducing two or more nucleic acid molecules that comprise portions of the polynucleotide construct into the plant or plant cell, wherein the two or more nucleic acid collectively comprise the polynucleotide construct. It is recognized that the two or more nucleic acid molecules can be recombined into the polynucleotide construct within a plant cell via homologous recombination methods that are known in the art.

Alternatively, the two or more nucleic acid molecules that comprise portions of the polynucleotide construct can be introduced a plant or cell thereof in a sequential manner. For example, a first nucleic acid molecule comprising a first portion of a polynucleotide construct can be introduced into a plant cell, and the transformed plant cell can then be regenerated into a plant comprising the first nucleic acid molecule. A second nucleic acid molecule comprising a second portion of a polynucleotide construct can then be introduced into a plant cell comprising the first nucleic acid molecule, wherein the first and second nucleic acid molecules are recombined into the polynucleotide construct via homologous recombination methods.

Methods of homologous recombination involve inducing double breaks in DNA using zinc-finger nucleases or homing endonucleases that have been engineered to make double-strand breaks at specific recognition sequences in the genome of a plant, other organism, or host cell. See, for example, Durai et al., (2005) Nucleic Acids Res 33:5978-90; Mani et al. (2005) Biochem Biophys Res Comm 335:447-57; U.S. Pat. Nos. 7,163,824, 7,001,768, and 6,453,242; Arnould et al. (2006) J Mol Biol 355:443-58; Ashworth et al., (2006) Nature 441:656-9; Doyon et al. (2006) J Am Chem Soc 128:2477-84; Rosen et al., (2006) Nucleic Acids Res 34:4791-800; and Smith et al. (2006) Nucleic Acids Res 34:e149; U.S. Pat. App. Pub. No. 2009/0133152; and U.S. Pat. App. Pub. No. 2007/0117128; all of which are herein incorporated in their entirety by reference.

TAL effector nucleases can also be used to make double-strand breaks at specific recognition sequences in the genome of a plant for gene modification or gene replacement through homologous recombination. TAL effector nucleases are a new class of sequence-specific nucleases that can be used to make double-strand breaks at specific target sequences in the genome of a plant or other organism. TAL effector nucleases are created by fusing a native or engineered TAL effector, or functional part thereof, to the catalytic domain of an endonuclease, such as, for example, FokI. The unique, modular TAL effector DNA binding domain allows for the design of proteins with potentially any given DNA recognition specificity. Thus, the DNA binding domains of the TAL effector nucleases can be engineered to recognize specific DNA target sites and thus, used to make double-strand breaks at desired target sequences. See, WO 2010/079430; Morbitzer et al. (2010) PNAS 10.1073/pnas.1013133107; Scholze & Boch (2010) Virulence 1:428-432; Christian et al. Genetics (2010) 186:757-761; Li et al. (2010) Nuc. Acids Res. (2010) doi:10.1093/nar/gkq704; and Miller et al. (2011) Nature Biotechnology 29:143-148; all of which are herein incorporated by reference.

For the transformation of plants and plant cells, the nucleotide sequences of the invention are inserted using standard techniques into any vector known in the art that is suitable for expression of the nucleotide sequences in a plant or plant cell. The selection of the vector depends on the preferred transformation technique and the target plant species to be transformed.

Methodologies for constructing plant expression cassettes and introducing foreign nucleic acids into plants are generally known in the art and have been previously described. For example, foreign DNA can be introduced into plants, using tumor-inducing (Ti) plasmid vectors. Other methods utilized for foreign DNA delivery involve the use of PEG mediated protoplast transformation, electroporation, microinjection whiskers, and biolistics or microprojectile bombardment for direct DNA uptake. Such methods are known in the art. (U.S. Pat. No. 5,405,765 to Vasil et al.; Bilang et al. (1991) Gene 100: 247-250; Scheid et al., (1991) Mol. Gen. Genet., 228: 104-112; Guerche et al., (1987) Plant Science 52: 111-116; Neuhause et al., (1987) Theor. Appl Genet. 75: 30-36; Klein et al., (1987) Nature 327: 70-73; Howell et al., (1980) Science 208:1265; Horsch et al., (1985) Science 227: 1229-1231; DeBlock et al., (1989) Plant Physiology 91: 694-701; Methods for Plant Molecular Biology (Weissbach and Weissbach, eds.) Academic Press, Inc. (1988) and Methods in Plant Molecular Biology (Schuler and Zielinski, eds.) Academic Press, Inc. (1989). The method of transformation depends upon the plant cell to be transformed, stability of vectors used, expression level of gene products and other parameters.

Other suitable methods of introducing nucleotide sequences into plant cells and subsequent insertion into the plant genome include microinjection as Crossway et al. (1986) Biotechniques 4:320-334, electroporation as described by Riggs et al. (1986) Proc. Natl. Acad. Sci. USA 83:5602-5606, Agrobacterium-mediated transformation as described by Townsend et al., U.S. Pat. No. 5,563,055, Zhao et al., U.S. Pat. No. 5,981,840, direct gene transfer as described by Paszkowski et al. (1984) EMBO J. 3:2717-2722, and ballistic particle acceleration as described in, for example, Sanford et al., U.S. Pat. No. 4,945,050; Tomes et al., U.S. Pat. No. 5,879,918; Tomes et al., U.S. Pat. No. 5,886,244; Bidney et al., U.S. Pat. No. 5,932,782; Tomes et al. (1995) “Direct DNA Transfer into Intact Plant Cells via Microprojectile Bombardment,” in Plant Cell, Tissue, and Organ Culture: Fundamental Methods, ed. Gamborg and Phillips (Springer-Verlag, Berlin); McCabe et al. (1988) Biotechnology 6:923-926); and Lec1 transformation (WO 00/28058). Also see, Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In Vitro Cell Dev. Biol. 27P:175-182 (soybean); Singh et al. (1998) Theor. Appl. Genet. 96:319-324 (soybean); Datta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); Tomes, U.S. Pat. No. 5,240,855; Buising et al., U.S. Pat. Nos. 5,322,783 and 5,324,646; Tomes et al. (1995) “Direct DNA Transfer into Intact Plant Cells via Microprojectile Bombardment,” in Plant Cell, Tissue, and Organ Culture: Fundamental Methods, ed. Gamborg (Springer-Verlag, Berlin) (maize); Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; Bowen et al., U.S. Pat. No. 5,736,369 (cereals); Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, New York), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); all of which are herein incorporated by reference.

The polynucleotides of the invention may be introduced into plants by contacting plants with a virus or viral nucleic acids. Generally, such methods involve incorporating a polynucleotide construct of the invention within a viral DNA or RNA molecule. It is recognized that the a protein of the invention may be initially synthesized as part of a viral polyprotein, which later may be processed by proteolysis in vivo or in vitro to produce the desired recombinant protein. Further, it is recognized that promoters of the invention also encompass promoters utilized for transcription by viral RNA polymerases. Methods for introducing polynucleotide constructs into plants and expressing a protein encoded therein, involving viral DNA or RNA molecules, are known in the art. See, for example, U.S. Pat. Nos. 5,889,191, 5,889,190, 5,866,785, 5,589,367 and 5,316,931; herein incorporated by reference.

In specific embodiments, the nucleotide sequences of the invention can be provided to a plant using a variety of transient transformation methods. Such transient transformation methods include, but are not limited to, the introduction of the nucleotide sequence or variants and fragments thereof directly into the plant. Such methods include, for example, microinjection or particle bombardment. See, for example, Crossway et al. (1986) Mol Gen. Genet. 202:179-185; Nomura et al. (1986) Plant Sci. 44:53-58; Hepler et al. (1994) Proc. Natl. Acad. Sci. 91: 2176-2180 and Hush et al. (1994) The Journal of Cell Science 107:775-784, all of which are herein incorporated by reference. Alternatively, the nucleotide sequence can be transiently transformed into the plant using techniques known in the art. Such techniques include viral vector system and Agrobacterium tumefaciens-mediated transient expression as described below.

The cells that have been transformed may be grown into plants in accordance with conventional ways. See, for example, McCormick et al. (1986) Plant Cell Reports 5:81-84. These plants may then be grown, and either pollinated with the same transformed strain or different strains, and the resulting hybrid having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present invention provides transformed seed (also referred to as “transgenic seed”) having a polynucleotide construct of the invention, for example, an expression cassette of the invention, stably incorporated into their genome.

The present invention may be used for transformation of any plant species, including, but not limited to, monocots and dicots. Examples of plant species of interest include, but are not limited to, peppers (Capsicum spp; e.g., Capsicum annuum, C. baccatum, C. chinense, C. frutescens, C. pubescens, and the like), tomatoes (Lycopersicon esculentum), tobacco (Nicotiana tabacum), eggplant (Solanum melongena), petunia (Petunia spp., e.g., Petunia×hybrida or Petunia hybrida), corn or maize (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers. Citrus spp. include, but are not limited to, cultivated citrus species, such as, for example, orange, lemon, meyer lemon, lime, key lime, Australian limes, grapefruit, mandarin orange, clementine, tangelo, tangerine, kumquat, pomelo, ugh, blood orange, citron, Buddha's hand, and bitter orange.

As used herein, the term “plant” includes plant cells, plant protoplasts, plant cell tissue cultures from which plants can be regenerated, plant calli, plant clumps, and plant cells that are intact in plants or parts of plants such as embryos, pollen, ovules, seeds, leaves, cotyledons, flowers, stems, shoots, hypocotyls, epicotyls, branches, fruits, roots, root tips, buds, anthers, scions, rootstocks, and the like. The present invention encompasses all plants derived from the regenerated plants of invention provided that these derived plants comprise the introduced polynucleotides. Such derived plants can also be referred to herein as derivative plants or derivatives.

The derivative plants or derivatives include, for example, sexually and asexually produced progeny, variants, mutants, and other derivatives of the regenerated plants that comprise at least one of the polynucleotides of the present invention. Also within the scope of the present invention are vegetatively propagated plants including, for example, plants regenerated by cell or tissue culture methods from plant cells, plants tissues, plant organs, other plant parts, or seeds, plants produced by rooting a stem cutting, and plants produced by grafting a scion (e.g., a stem or part thereof, or a bud) onto a rootstock which is the same species as the scion or a different species. Stich vegetatively propagated plants or at least one part thereof comprise at least one polynucleotide of the present invention. It is recognized that vegetatively propagated plants are also known as clonally propagated plants, asexually propagated, or asexually reproduced plants.

The invention is drawn to compositions and methods for increasing resistgance to plant disease. By “disease resistance” is intended that the plants avoid the disease symptoms that are the outcome of plant-pathogen interactions. That is, pathogens are prevented from causing plant diseases and the associated disease symptoms, or alternatively, the disease symptoms caused by the pathogen are minimized or lessened.

Pathogens of the invention include, but are not limited to, bacteria that are known to cause ACC and other forms of citrus canker caused by Xanthomonas strains, such as, for example, the Xanthomonas strains disclosed herein.

The invention provides host cells comprising at least one polynucleotide construct or nucleic acid molecule of the present invention. Such host cells include, for example, bacterial cells, fungal cells, animal cells, and plant cells. Preferably, the host cells are non-human, host cells. More preferably, the host cells are plant cells. Additionally, the invention encompasses viruses and viroids comprising at least one polynucleotide construct or nucleic acid molecule of the present invention.

The following examples are offered by way of illustration and not by way of limitation.

### Example

Based on recent findings predicting activation of the UPT boxes by TAL effectors and the fact that at least one significant TAL effector, PthA, is present in X. citri and critical for virulence, it was hypothesized that engineering a super promoter which contains several putative UPT boxes fused to an “execution” (cell death triggering) gene could be used to target PthA and other prevalent AvrBs3 homolog proteins in X. citri that when injected by the bacterium into the plant cell would activate the execution gene producing an HR. AvrGf1 from X. citri strain A^{w} was selected (Rybak et al. (2009) Mol. Plant Pathol. 10:249-262) as the execution gene because of its ability to elicit an HR in grapefruit upon delivery into plant cells. A transient assay in grapefruit (Citrus paradisi) was developed to test constructs for this resistance approach. The assay entails transforming grapefruit leaves with Agrobacterium tumefaciens containing a T-DNA construct comprised of a Bs3 promoter construct fused to the execution gene, avrGf1, followed by co-inoculating the same leaf area with X. citri strains and assessing the reaction. Transient assays demonstrated that an HR could indeed be generated by specific interactions between TAL effectors and particular UPT boxes in Bs3 promoter constructs. Additionally we have demonstrated that stable transgenic grapefruit plants transformed with Bs3 promoter constructs fused to avrGf1 show resistance against X. citri strains.

### Bs3 Promoter Constructs can be Triggered in Grapefruit Leaves by TAL Effectors Delivered Through the Type III Secretion System.

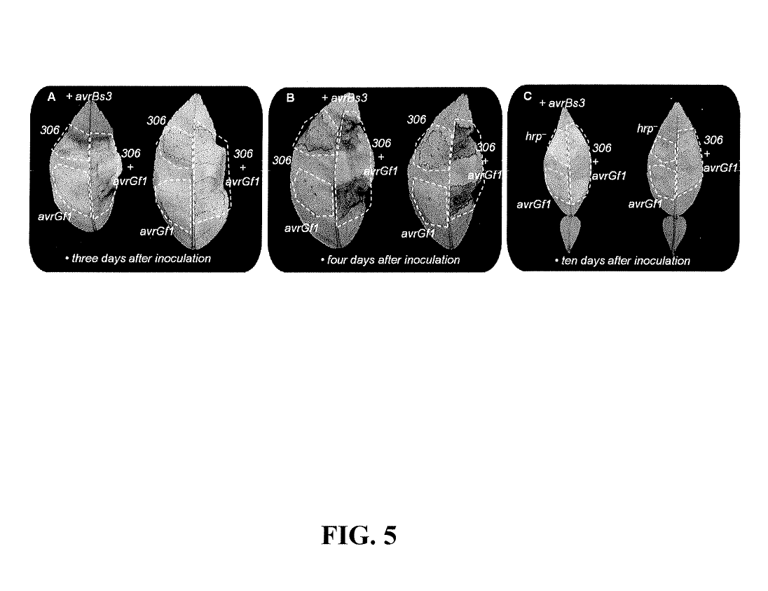

To test if the pepper Bs3 promoter functions in grapefruit, young leaves were transiently transformed with A. tumefaciens containing the binary vector pKBs3::avrGf1 (31+Bs3::avrGf1), which contains the avrGf1 gene under the control of the Bs3 native promoter, and were later assessed alone or in conjunction with bacterially delivered TAL effectors. TAL effector delivery was carried out by co-inoculating leaf areas with X. citri strain 306 (Xcc-306) or Xcc-306 expressing avrBs3 (306+avrBs3). After three days leaves were examined for their reaction to bacterial strains. No reaction was apparent in leaf areas inoculated with only the Bs3::avrGf1 construct (FIG. 5A-B, lower left leaf areas). Leaf areas infiltrated with Xcc-306 or Xcc-306+avrbs3 in the absence of the avrGf1 construct produced citrus canker symptoms indicative of a disease reaction (FIG. 5A-B, upper left leaf areas). However when leaves were infiltrated with Xcc-306+avrbs3 in the presence of the avrGf1 construct, an HR was visible within three days and more strongly at four days (FIG. 5. A-B, right areas of first leaf in each photo). An HR did arise in the presence of Xcc-306 however it was not strongly visible till four days after Xcc-306 inoculation (FIG. 5A-B, right areas of second leaf in each photo). We concluded that the specific interaction between AvrBs3 and the UPA box in the Bs3 promoter produced a strong induction of the avrGf1 gene, and an unexpected weaker induction was triggered by one of the native TAL effectors in Xcc-306. To confirm that the HR was triggered by effectors delivered via the T3SS, we performed the same assay using a T3SS defective strain with a mutation in the hrpG locus (3060 hrpG) (Wengelnik et al. 1996). Co-infiltration of 31+Bs3::avrGf1 strain with either, 306ΩhrpG or 306ΩhrpG+avrBs3 resulted in no observable HR on grapefruit leaves (FIG. 5C), and neither strain could incite citrus canker symptoms in the absence of the resistance constructs. This result demonstrates the dependence on T3-secreted TAL effectors for both virulence and resistance reactions.

### Specificity of Bs3 Promoter Induction.

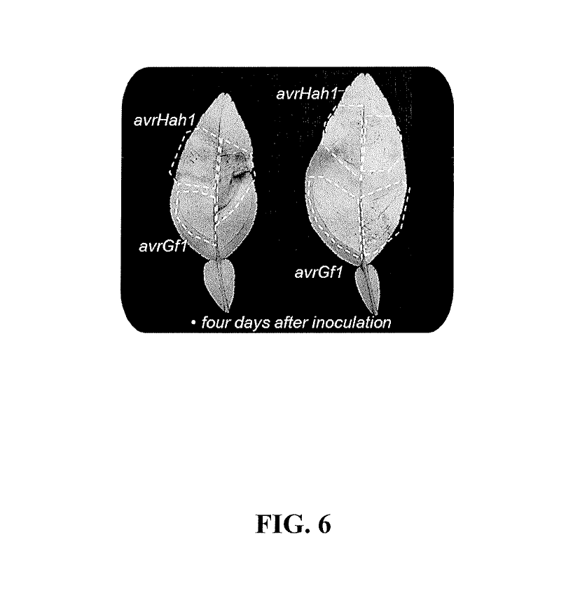

To examine the specificity of induction of the Bs3 promoter, we investigated the ability of AvrHah1, a TAL effector from Xanthomonas gardeneri with the same DNA binding specificity as AvrBs3 (Schornack et al. (2008) New Phytol. 179:546-566), to activate our Bs3 construct. Agrobacterium carrying the Bs3 native promoter construct was infiltrating into grapefruit leaves and later X. gardneri strains with or without avrHah1 were co-inoculated onto the same leaf areas. Both the native X. gardneri strain and the avrHah1⁻ mutant produced mild reactions on grapefruit leaves (FIG. 6) likely due to other effectors in this strain. In the absence of bacterial effectors, the avrGf1 construct produced no reaction, however in combination with X. gardneri, an HR was evident at four days after infiltration (FIG. 6). In contrast, the X. gardneri avrHah1⁻ mutant did not produce an HR in the presence of the avrGf1 construct (FIG. 6).

We have observed that the wild type X. citri strain 306 can activate the Bs3 promoter (FIG. 5A-B) in the absence of AvrBs3, so we investigated which of the four native X. citri TAL effectors may give rise to this reaction. It is known that PthA4 in Xcc 306 and its homologs in other strains is the key TAL effector for virulence (Al-Saadi, et al. 2007), therefore we generated the strain 306ΔpthA4 mutant, which carries a deletion in the pthA4 gene leaving pthA1-3 intact. When co-inoculated with the native Bs3::avrGf1 construct, this mutant did not result in an HR indicating that PthA4 is involved in triggering this promoter construct. There is overlap in the binding specificity of AvrBs3 and PthA4, thus it is possible that PthA4 triggers the resistance promoter via the UPA box.

Both of these results demonstrate that the activation of the Bs3 promoter is specific for TAL effectors with RVDs that recognize DNA sequences in the Bs3 promoter.

A Bs3 Super Promoter Shows Robust Activity in Grapefruit Cells Towards TAL-Effectors from Diverse X. Citri Isolates.

Previously we have shown that the Bs3 promoter can be engineered to contain multiple UPT boxes to confer activation by a number of disparate TAL effectors (Römer et al. (2009) PNAS 106:20526-20531). In an attempt to broaden the range of citrus canker resistance with the native Bs3 promoter construct, we engineered a new promoter named the Bs3₁₄ₓ super promoter that contains 14 different UPT boxes. These 14 different UPT boxes were designed based on the TAL effector code (Boch et al. (2009) Science 326:1509-1512) as recognition sites for seventeen of the reported X. citri TAL-effectors (Table 2). The Bs3₁₄ₓ super promoter further comprises the UPA box (also known as UPT_{AvrBs3}) that is the recognition site for AvrBs3.

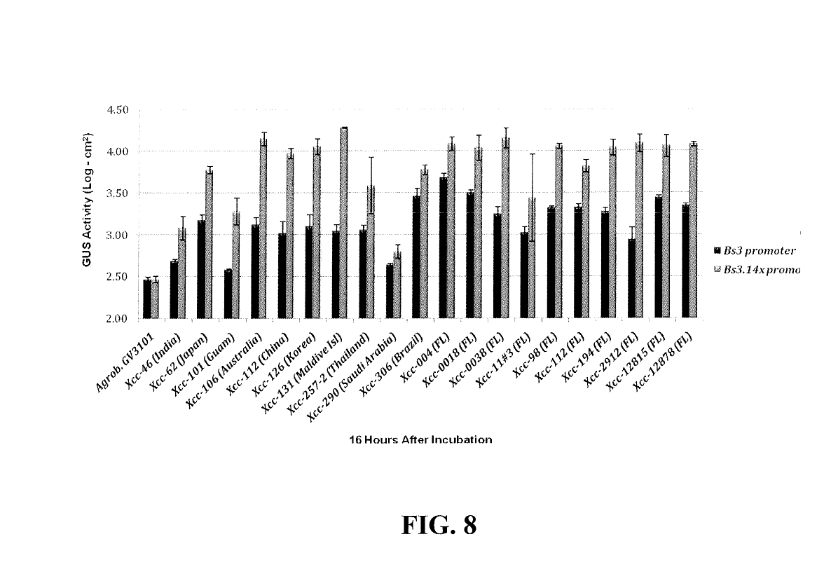

We used the Bs3₁₄ₓ::avrGf1 super promoter construct to test the recognition of TAL effectors in more than twenty X. citri strains collected worldwide and derivatives. Co-inoculations of the super promoter resistance construct together with each of the strains demonstrated that the Bs3 super promoter is triggered by a broad range of X. citri strains (Table 5, FIG. 8). Notable exceptions were two strains, X. citri strain 101 isolated in Guam and X. citri strain 290 from Saudi Arabia which both failed to cause disease symptoms on susceptible leaves. On leaves transformed with the disease cassette, strain 101, but not strain 290, induced HR. We attempted to complement these strains with the TAL effectors AvrBs3 or PthAw (pthA^{w}5.2). Whereas the addition of AvrBs3 did not enable strain 101 to cause citrus canker symptoms, PthA^{w}5.2 did confer virulence in strain 101. In strain 290, PthA^{w}5.2 could neither restore virulence nor trigger a resistance reaction. We also tested complementation in the strain X. citri 306ΔpthA4 with either PthA^{w}5.2 or AvrTaw (Table 5). Our results showed that PthA^{w}5.2 could complement PthA4 function and confer disease on susceptible leaves, whereas AvrTaw could not. These studies demonstrate that the super promoter construct can confer broad resistance to a large number of strains, that the two atypical strains have defects in either primary TAL effectors (101) or effector production or secretion (290), and that PthA^{w}5.2 can functionally replace the virulence activity of PthA4 but AvrTaw can not.

TABLE 5
Survey of the Reaction of Worldwide Xanthomonas citri Isolates on
Grapefruit Leaves in the Presence or Absence of a Resistance Construct.

| | | Disease Reaction^{a} | |
|---|---|---|---|
| Strains Designation | Origin | Susceptible | Resistant |
| X. citri-306 | Brazil | Disease | HR^{c} |
| X. citri-306 + avrBs3 | this study | Disease | HR |
| 306ΩhrpG | this study | No reaction | Not tested |
| 306ΔpthA4 | this study | No reaction | HR |
| 306ΔpthA4 + pthA^{W}5.2 | this study | Disease | HR |
| 306ΔpthA4 + avrTaw | this study | No reaction | HR |
| X. citri-101 | Guam | No reaction | HR |
| X. citri-101 + avrBs3 | this study | No reaction | HR |
| X. citri-101 + pthA^{w}5.2 | this study | Disease | HR |
| X. citri-290 | Saudi Arabia | No reaction | No reaction |
| X. citri-290 + pthA^{w}5.2 | this study | No reaction | No reaction |
| X. citri-46 | India | Disease | HR |
| X. citri-62 | Japan | Disease | HR |
| X. citri-106 | Australia | Disease | HR |
| X. citri-112 | China | Disease | HR |
| X. citri-131 | Maldives Islands | Disease | HR |
| X. citri-126 | Korea | Disease | HR |
| X. citri-257-2 | Thailand | Disease | HR |
| X. citri-004 | Florida - USA | Disease | HR |
| X. citri-11-3 | Florida - USA | Disease | HR |
| X. citri-0018 | Florida - USA | Disease | HR |
| X. citri-0038 | Florida - USA | Disease | HR |
| X. citri-98 | Florida - USA | Disease | HR |
| X. citri-00112 | Florida - USA | Disease | HR |
| X. citri-00194 | Florida - USA | Disease | HR |
| X. citri-02912 | Florida - USA | Disease | HR |
| X. citri-12815 | Florida - USA | Disease | HR |
| X. citri-12870 | Florida - USA | Disease | HR |

^{a}Strains were inoculated onto grapefruit leaves in the absence (Susceptible) or presence (Resistant) of the transiently transformed Bs3₁₄ₓ::avrGf1 resistance construct;
^{c}(HR) hypersensitive reaction. The resistance construct was introduced by Agrobacterium transformation; Agrobacterium inoculation alone produced no reaction on grapefruit leaves.

Population Dynamics of X. Citri Subsp Citri in Grapefruit Transiently Transformed with Bs3::avrGf1.

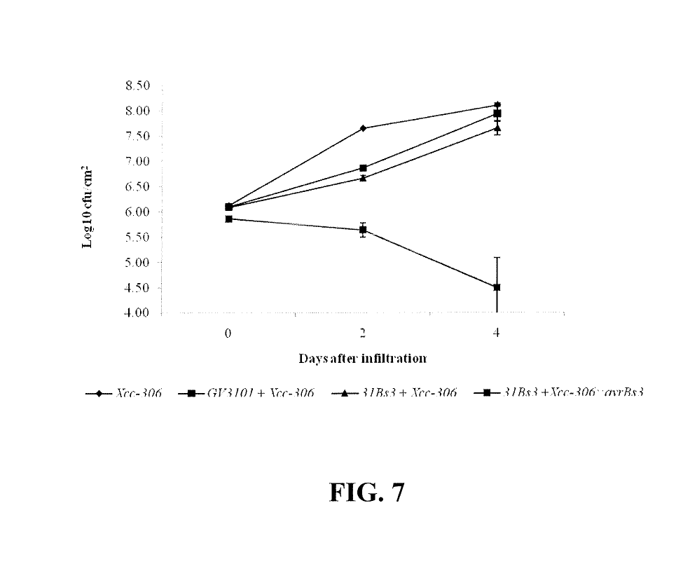

To substantiate that the observed cell death triggered by the interaction between the Bs3 native promoter and AvrBs3 was a bona fide HR, in planta bacterial populations were monitored over a four-day period. Grapefruit leaves were infiltrated with different combinations of X. citri strains and Agrobacterium with and without the resistance construct, and the X. citri populations were assessed after three days. Populations of Xcc-306 alone grew by about 2 logs, as did Xcc-306 co-inoculated with Agrobacterium lacking the resistance cassette (FIG. 7). Populations of Xcc-306 were slightly lower in the presence of the resistance cassette, growing only about 1.5 logs, however the specific combination of Xcc-306+avrBs3 with the Bs3::avrGf1 construct strongly suppressed X. citri growth below the initial inoculum level, resulting in a 3 log difference compared to the other strains (FIG. 7). Therefore the Bs3 promoter construct is conferring effective resistance to citrus canker.

Bs3₁₄ₓ Super Promoter Showed Higher Induction by TAL-Effectors Compared with the Bs3 Single Promoter.

To quantify the induction of the Bs3 promoter constructs, we generated additional T-DNA constructs of the native Bs3 and Bs3₁₄ₓ super promoter fused to the reporter gene, GUS. The GUS constructs were delivered transiently into grapefruit leaves in Agrobacterium (31+Bs3::GUS and Bs3::GUS₁₄ₓ), which were subsequently co-inoculated with twenty of the X. citri strains listed in Table 5. We determined the level of gene expression quantitatively using the GUS assay to compare in vivo promoter activity between the native Bs3 promoter with just the UPT_{AvBs3} box and the Bs3₁₄ₓ super promoter. Analysis of the Bs3 native promoter showed that several of the Florida X. citri strains, (Xcc-004, Xcc-0018, Xcc-12815, Xcc-12878) and the Brazilian strains Xcc-306 had higher GUS activity compared with the other X. citri strains tested (FIG. 8). These differences were much less with the Bs3₁₄ₓ super promoter which showed higher activity overall, likely due to activation through multiple UPT boxes by additional TAL effectors. No significant GUS activity was observed with the Guam (101) and Saudi Arabian (290) strains, consistent with HR results in Table 5. The Guam strain did show higher GUS activity in the presence of the Bs3₁₄ₓ super promoter indicating that it may be able to deliver other TAL effectors that can trigger the Bs3₁₄ₓ super promoter. Additionally, X. citri, strain 46 from India showed a low level of activity, however this strain behaved typically in pathogen tests (Table 5). These results confirm that the Bs3_{14X} super promoter is effectively activated by a wide range of citrus strains to a high level.

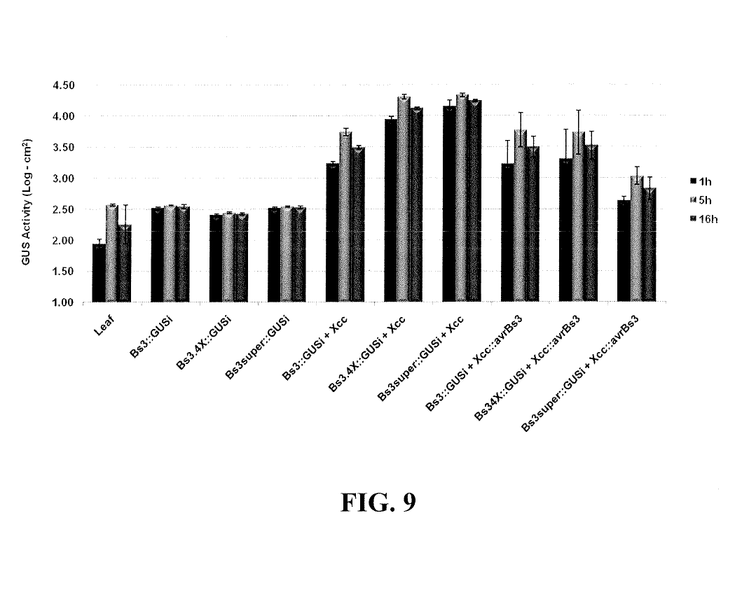

To be sure we were not measuring GUS activity expressed in the Agrobacterium cells used for transformation, we also assessed GUS activity driven by both Bs3 promoters using the GUS-intron reporter gene (GUSi) that is expressed only in plant cells. The GUS activity level measured in grapefruit leaves transiently transformed with Agrobacterium containing either the Bs3 native or Bs3₁₄ₓ super promoter GUS constructs in the absence of X. citri strains showed comparable levels of GUS activity to non-inoculated leaves (FIG. 9). In the presence of Xcc-306, GUS activity increased in leaves with the native Bs3 promoter and to higher levels with the Bs3₁₄ₓ super promoter. GUS activity was also increased with Xcc-306+AvrBs3 but to a lesser degree and with a smaller difference in overall levels. The absence of GUS activity in the absence of X. citri and the fact that the levels of GUS activity observed in this experiment are comparable to levels of GUS activity in previous experiments using the standard GUS reporter gene demonstrates that we are not measuring spurious GUS activity in Agrobacterium cells.

Our previous experience engineering Bs3 promoters with additional UPT boxes was limited to three UPT boxes, and these showed tight regulation. In the current work, we were uncertain if 14 UPT boxes could also retain tight regulation, so we designed a shorter super promoter construct with four UPT boxes targeting a core set of ten citrus TAL effectors. This promoter is referred to as the Bs3_{4X} short promoter and is comprised of the following UPT boxes: UPT_{Apl1} (SEQ ID NO: 5), UPT_{pB3.7} (SEQ ID NO: 15), UPT_{Apl2} (SEQ ID NO: 6), and UPT_{AvrTAw} (TATAACACCCTCAACATAAT; SEQ ID NO: 19). Testing of this promoter fused to the GUSi reporter gene demonstrated that it was activated comparably to the Bs3₁₄ₓ super promoter (FIG. 9).

### Pathogen Testing in Stable Transgenic Grapefruit Lines Demonstrates Citrus Canker Resistance.

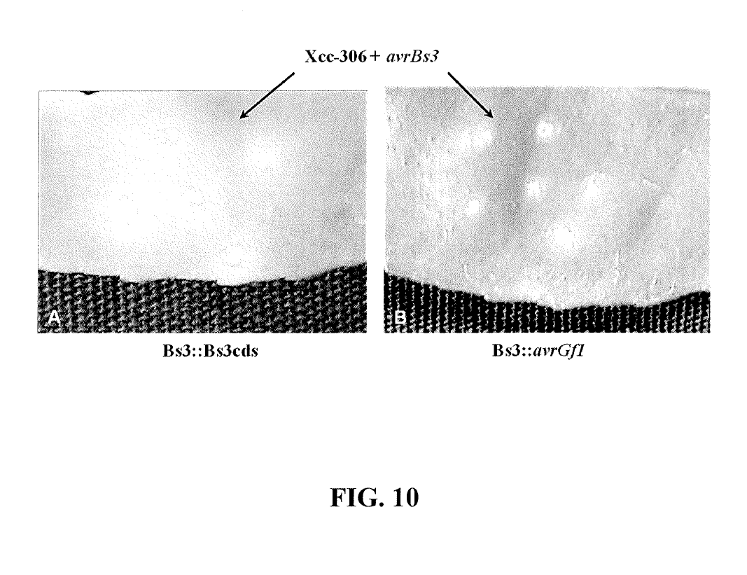

Pathogen challenge of stable transgenic lines was carried out by standard pin-prick inoculation of young transgenic grapefruit plants. Plants transformed with Bs3::avrGf1 were challenged with Xcc.306+avrBs3. Several independent primary transformed lines were assessed after 28 days and showed no canker lesions or yellow discoloration around the sites of inoculation, typical of citrus canker disease, (FIG. 10). Instead, there were localized areas of necrosis consistent with a hypersensitive resistance response. In contrast, other transgenic lines transformed with a different construct using the Bs3 promoter fused to the Bs3 coding sequence did show raised lesions and yellowing typical of a susceptible reaction. Although the Bs3 coding sequence does encode a plant execution gene, it appears to work weakly or not at all in these assays or mutations may occur in the coding sequence of these lines.

### Material and Methods

### Bacterial Strains and Plasmids.

The bacterial strains and plasmids used in this example are listed in Table 6.

TABLE 6
Bacterial Strains and Plasmids

| | | |
|---|---|---|
| Strain or Construct | Relevant characteristics | Source |

X. citri subsp. citri

| | | |
|---|---|---|
| Xcc.306 | Wild-type, strain A, isolated in Brazil, Rif^{r} | DPI¹ |
| 306ΩhrpG | hrcC⁻, single recombinant of pCRhrcC, Km^{r} | This study |
| Xcc-46 | Wild-type, strain A, isolated in India | DPI |
| Xcc-62 | Wild-type, strain A, isolated in Japan | DPI |
| Xcc-101 | Wild-type, strain A*, isolated in Guam | DPI |
| Xcc-106 | Wild-type, strain A, isolated in Australia | DPI |
| Xcc-126 | Wild-type, strain A, isolated in China | DPI |
| Xcc-131 | Wild-type, strain A, isolated in Maldive Island | DPI |
| Xcc-252-2 | Wild-type, strain A, isolated in Thailand | DPI |
| Xcc-290 | Wild-type, strain A*, isolated in Saudi Arabia | DPI |
| Xcc-004 | Wild-type, strain A, isolated in Florida, USA | DPI |
| Xcc-0018 | Wild-type, strain A, isolated in Florida, USA | DPI |
| Xcc-0038 | Wild-type, strain A, isolated in Florida, USA | DPI |
| Xcc-11#3 | Wild-type, strain A, isolated in Florida, USA | DPI |
| Xcc-98 | Wild-type, strain A, isolated in Florida, USA | DPI |
| Xcc-112 | Wild-type, strain A, isolated in Florida, USA | DPI |
| Xcc-194 | Wild-type, strain A, isolated in Florida, USA | DPI |
| Xcc-2919 | Wild-type, strain A, isolated in Florida, USA | DPI^{a} |
| Xcc-12815 | Wild-type, strain A, isolated in Florida, USA | DPI |
| Xcc-12878 | Wild-type, strain A, isolated in Florida, USA | DPI |

X. gardineri

| | | |
|---|---|---|
| XV444 | HR(+) ECW-30R, avrHah1(+) | Schornack et al. |
| | | (2008) |
| 1782 | HR(−) ECW-30R, avrHah1(−) | Schornack et al. |
| | | (2008) |

Agrobacterium tumefaciens

| | | |
|---|---|---|
| GV3101 | Disarmed A. tumefaciens strain, Rif^{r} | Van Larebeke et |
| | | al. 1974 |

Escherichia coli

| | | |
|---|---|---|
| DH5α | F⁻ recA hsdR17 (rk-mk+) φ80dLacZ | Bethesda |
| | | Research |
| | | Laboratories |

Plasmids

| | | |
|---|---|---|
| pLAFR3 | Tra⁻Mob⁺, RK2 replicon; tet^{r} | Staskawicz et |
| | | al. 1987 |
| pUFR034 | Inc W, Mob+, lacZα, Par⁺, cosmid; Km^{r} | De Feyter et al. |
| | | 1990 |
| pRK2073 | Sp^{r} Tra⁺, helper plasmid; Sm^{r} | Figurski and |
| | | Helinski 1979 |
| pENTR/D-TOPO | Entry vector for Gateway ® technology | Invitrogen |
| pGWB2 | Binary expression vector, contains 35S | Nakagawa et al. |
| | promoter upstream of attR1-Cm^{r}-ccdB-attR2; | (2007) |
| | Hm^{r}, Km^{r} | |
| pGWB3 | Binary expression vector, contains attR1-Cm^{r}- | Nakagawa et al. |
| | ccdB-attR2-GUS; Hm^{r}, Km^{r} | (2007) |
| pGWB5 | binary gfp expression vector, contains attR1- | Nakagawa et al. |
| | Cm^{r}-ccdB-attR2-sgfp; Hm^{r}, Km^{r} | (2007) |
| pK7-GW-Gs3 | Derivative of pK7FWG2 containing Bs3_{cds} | Römer et al. |
| | | 2009 |
| pK7-GUSi | Derivative of pK7-GW-Gs3 containing GUS- | This study |
| | intron gene | |

Constructs²

| | | |
|---|---|---|
| pL799 | pLAFR3 with DNA fragment from Xac-A^{w} | Rybak et al., |
| | that contains avrGf1 | 2009 |
| pLAT211 | pLAFR3 containing avrBs3-2 | Bonas et al., |
| | | 1991 |
| pAW5.2 | pthA^{W} from X. citri strain X0053 | Al-Saadi et al., |
| | | 2007 |
| pUFR80.1 | avrBs3 homolog from X. citri strain A^{w}, | Rybak et al., |
| | designated avrTaw | 2009 |
| pGavrGf1_2 | Derivative of pGWB2 containing avrGf1 | This study |
| pGavrGf1_5 | Derivative of pGWB5 containing avrGf1 | This study |
| pGBs3::GUS | Derivative of pGWB3 containing Bs3 | This study |
| | promoter | |
| pGBs3₄ₓ::GUS | Derivative of pGWB3 containing Bs3₄ₓ | This study |
| | promoter (four UPT box) | |
| pGBs3₁₄ₓ::GUS | Derivative of pGWB3 containing Bs3₁₄ₓ | This study |
| | promoter (14 UPT box) | |
| pK7Bs3::avrGf1 | Derivative of pK7-GW-Gs3 containing avrGf1 | This study |
| | drive by Bs3 promoter | |
| pK7Bs3₁₄ₓ::avrGf1 | Derivative of pK7-GW-Gs3 containing avrGf1 | This study |
| | drive by Bs3 super promoter (14 UPT box) | |
| pK7Bs3::GUSi | Derivative of pK7-GUSi containing Bs3 | This study |
| | promoter | |
| pK7Bs3₄ₓ::GUSi | Derivative of pK7-GUSi containing Bs3₄ₓ | This study |
| | promoter (four UPT box) | |
| pK7Bs3₁₄ₓ::GUSi | Derivative of pK7-GUSi containing Bs3₁₄ₓ | This study |
| | promoter (14 UPT box) | |

¹Division of Plant Industry (DPI), Gainesville, Florida, USA.
²Constructs were generated through standard cloning methods as previously described in Romer et al. (2009) PNAS 106: 20526-20531.

### Plant Material and Plant Inoculations.

Plants used in this study include Grapefruit cv. Duncan (Citrus paradisi) and the transgenic lines generated by using the Bs3 promoter system. The plants were grown in the glasshouse at temperatures ranging from 25-30° C. Young leaves were used for inoculations based on the following scale: young leaves (two to three week-old leaves after the pruning), intermediate aged leaves (three to five week-old leaves after the pruning) and old leaves (five or more week-old leaves after pruning). For infiltration, three week-old leaves were inoculated with bacterial suspensions via a hypodermic needle and syringe into the abaxial surface of the leaf. For the preparation of bacterial suspensions of Xanthomonas strains, 18 h cultures were harvested from solid medium, suspended in sterile tap water, and standardized to an optical density (OD₆₀₀) of 0.3 (5×10⁸ colony-forming units (cfu) ml⁻¹).

### Pathogen-Induced Cell Death Assay.

For the induction of cell death, the Bs3 native promoter or the Bs3₁₄ₓ super promoter:avrGf1 constructs were transiently transformed in intact grapefruit leaf. Briefly, A. tumefaciens harboring the desired constructs were infiltrated into grapefruit leaves, and the same infiltrated areas were co-inoculated five hours later with X. citri suspensions. The plants were maintained in the growth room at 28° C. and monitored for HR symptoms for up to 10 days.

Measurement of Xanthomonas citri Survival in Transiently Transformed Grapefruit.

For measurement of X. citri growth in planta, intact grapefruit leaves were inoculated and co-inoculated as described. At 0, 2, and 4 days after infiltration, bacterial populations were measured from each of three leaves. An infiltrated leaf disc (0.5 cm² diameter) was placed in 1 ml of sterile tap water and triturated. Ten-fold dilutions with sterile tap water were made and 50 μL were plated onto NA plates. Bacterial colonies were counted and populations were calculated. Experiments were repeated at least three times.

### Quantification of β-Glucuronidase (GUS) Activity.

The amounts of GUS were measured using the fluorescent substrate methylumbelliferyl glucuronate (MUG, Sigma) according to standard protocols (Jefferson (1987) Plant Mol. Biol. Rep. 5:387-405; Basim et al. (2005) Appl. Environ. Microbiol. 71:8284-8291), with some modifications. Three leaf discs were collected using a cork borer (1 cm² diameter), and placed in individual eppendorf tubes containing 400 μL of MUG solution. The disks were homogenized and incubate at 37° C. for up to 24 hours. The GUS activity was determined by measuring the fluorescence using a CytoFluor II fluorescence multiwall plate reader (PerSeptive Biosystems, Framingham, Mass.) in an interval of 1 h, 6 h and 18 h after incubation. The final results were the average of the readings converted to a log scale.

### Generation of Transgenic Grapefruit Lines.

Transformation of citrus was carried out as described (Luth and Moore (1999) Plant Cell Tiss. Org. Cult. 57:219-222). Briefly seeds of Citrus×paradisi cv. Duncan were sterilized and germinated. Epicotyl segments from etiolated in vitro grown seedlings were inoculated with Agrobacterium tumefaciens, co-cultivated for 2-3 days, and transferred to a shooting medium containing a selective agent. Shoots typically appeared after 3-5 weeks and were placed in an elongation medium for another 2-3 weeks before transfer to rooting medium. Following one to two months of rooting, plants were transferred to soil and analyzed by PCR assay and pathogenicity tests.

### Pathogenicity Assay.

Transgenic grapefruit plants were grown in the growth chamber until leaves were adequate size. Bacterial suspension at concentration of 5×10⁸ cfu/ml, were introduced locally by pin-prick inoculation over the adaxial leaf surface. Plants were maintained in the same condition as mentioned above and responses assessed over time period of 30 days.

The article “a” and “an” are used herein to refer to one or more than one (i.e., to at least one) of the grammatical object of the article. By way of example, “an element” means one or more element.

Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

1. A method for making a citrus plant with enhanced resistance to citrus canker, said method comprising:
(a) stably transforming at least one citrus plant cell with a polynucleotide construct comprising a promoter operably linked to a nucleotide sequence encoding an execution protein, wherein the promoter comprises at least one UPT box, and wherein the execution protein is capable of triggering cell death in a citrus plant; and
(b) regenerating a transformed citrus plant from the transformed citrus plant cell, wherein the transformed citrus plant comprises enhanced resistance to at least one Xanthomonas strain that causes citrus canker.

2. The method of claim 1, wherein the UPT box is capable of binding with at least one TAL effector from at least one Xanthomonas strain that causes citrus canker.

3-4. (canceled)

5. The method of claim 1, wherein the execution protein is AvrGf1.

6. (canceled)

7. The method of claim 1, wherein the promoter is the Bs3 promoter.

8. (canceled)

9. The method of claim 1, wherein the promoter is a modified Bs3 promoter comprising one or more of the UPT boxes set forth in Table 3.

10. (canceled)

11. The method of claim 9, wherein the modified Bs3 promoter is the Bs3₁₄ₓ super promoter comprising the nucleotide sequence set forth in SEQ ID NO: 2.

12. The method of claim 9, wherein the modified Bs3 promoter is the Bs3_{4X} short promoter comprising the nucleotide sequence set forth in SEQ ID NO: 3.

13. The method of claim 1, wherein the citrus plant is selected from the group consisting of orange, lemon, meyer lemon, lime, key lime, Australian limes, grapefruit, mandarin orange, clementine, tangelo, tangerine, kumquat, pomelo, ugli, blood orange, citron, Buddha's hand, and bitter orange.

14. A citrus plant comprising stably incorporated into its genome a polynucleotide construct comprising a promoter operably linked to a nucleotide sequence encoding an execution protein, wherein the promoter comprises at least one UPT box, and wherein the execution protein is capable of triggering cell death in a citrus plant.

15. The citrus plant of claim 14, wherein the UPT box is capable of binding with at least one TAL effector from at least one Xanthomonas strain that causes citrus canker.

16. (canceled)

17. The citrus plant of claim 14, wherein the citrus plant comprises enhanced resistance to at least one Xanthomonas strain that causes citrus canker.

18. (canceled)

19. The citrus plant of claim 14, wherein the execution protein is AvrGf1.

20. (canceled)

21. The citrus plant of claim 14, wherein the promoter is the Bs3 promoter.

22. (canceled)

23. The citrus plant of claim 14, wherein the promoter is a modified Bs3 promoter comprising one or more of the UPT boxes set forth in Table 3.

24. (canceled)

25. The citrus plant of claim 23, wherein the modified Bs3 promoter is the Bs3₁₄ₓ super promoter comprising the nucleotide sequence set forth in SEQ ID NO: 2.

26. The citrus plant of claim 23, wherein the modified Bs3 promoter is the Bs3_{4X} short promoter comprising the nucleotide sequence set forth in SEQ ID NO: 3.

27. The citrus plant of claim 14, wherein the citrus plant is selected from the group consisting of orange, lemon, meyer lemon, lime, key lime, Australian limes, grapefruit, mandarin orange, clementine, tangelo, tangerine, kumquat, pomelo, ugli, blood orange, citron, Buddha's hand, and bitter orange.

28. A derivative citrus plant of the citrus plant of claim 27, wherein the derivative citrus plant comprises the polynucleotide construct.

29. (canceled)

30. A nucleic acid molecule comprising a promoter operably linked to a nucleotide sequence encoding an execution protein, wherein the promoter comprises at least one UPT box, and wherein the execution protein is capable of triggering cell death in a citrus plant.

31. The nucleic acid molecule of claim 30, wherein the UPT box is capable of binding with at least one TAL effector from at least one Xanthomonas strain that causes citrus canker.

32. (canceled)

33. The nucleic acid molecule of claim 30, wherein the execution protein is AvrGf1.

34. (canceled)

35. The nucleic acid molecule of claim 30, wherein the promoter is the Bs3 promoter.

36. (canceled)

37. The nucleic acid molecule of claim 30, wherein the promoter is a modified Bs3 promoter comprising one or more of the UPT boxes set forth in Table 3.

38. (canceled)

39. The nucleic acid molecule of claim 37, wherein the modified Bs3 promoter is the Bs3₁₄, super promoter comprising the nucleotide sequence set forth in SEQ ID NO: 2.

40. The nucleic acid molecule of claim 37, wherein the modified Bs3 promoter is the Bs3_{4X} short promoter comprising the nucleotide sequence set forth in SEQ ID NO: 3.

41. An expression cassette, vector, or plant cell comprising the nucleic acid molecule of claim 30.

42. (canceled)

43. A nucleic acid molecule comprising a nucleotide sequence selected from the group consisting of:
(a) the nucleotide sequence set forth in SEQ ID NO: 2;
(b) the nucleotide sequence set forth in SEQ ID NO: 3;
(c) a nucleotide sequence that is a functional variant of (a) or (b);
(d) a nucleotide sequence comprising at least 90% nucleotide sequence identity to the full-length nucleotide sequence of (a), wherein the nucleotide sequence comprises the UPT boxes and promoter activity of (a); and
(e) a nucleotide sequence comprising at least 90% nucleotide sequence identity to the full-length nucleotide sequence of (b), wherein the nucleotide sequence comprises the UPT boxes and promoter activity of (b).

44. The nucleic acid molecule of 43, wherein the functional variant is inducible by the same TAL effectors as (a) or (b).

45. The nucleic acid molecule of claim 43, wherein the functional variant retains that promoter activity of (a) or (b).

46. The nucleic acid molecule of claim 43, wherein the nucleic acid molecule further comprises an operably linked polynucleotide encoding an execution protein.

47. The nucleic acid molecule of claim 46, wherein the execution protein is AvrGf1.

48. (canceled)

49. An expression cassette comprising the nucleic acid molecule of claim 43 operably linked to a coding sequence.

50. (canceled)

51. A plant, plant cell, or host cell comprising the nucleic acid molecule of claim 43.

52. A method of producing citrus fruit comprising growing at least one citrus plant of claim 14 under conditions favorable for the growth of, and fruit production, by the citrus plant.
